(19) United States
(12) Patent Application Publication
(10) Pub. No.: US 2025/0374817 A1
(43) Pub. Date: Dec. 4, 2025

(54) METHOD FOR REFINING ORGANIC MATERIAL

(71) Applicant: Samsung Display Co., Ltd., Yongin-si, Gyeonggi-do (KR)

(72) Inventor: KEUNHEE HAN, Yongin-si, Gyeonggi-do (KR)

(73) Assignee: Samsung Display Co., Ltd., Yongin-si, Gyeonggi-do (KR)

(21) Appl. No.: 19/223,178

(22) Filed: May 30, 2025

(30) Foreign Application Priority Data
May 31, 2024 (KR) 10-2024-0071987

(51) Int. Cl. H10K 71/00; B01D 15/18; C07C 253/34; C09K 11/06; H10K 50/12; H10K 59/12; H10K 59/122; H10K 85/60

(52) U.S. Cl. CPC H10K 71/311; B01D 15/1871; C07C 253/34; C09K 11/06; H10K 59/1201; H10K 85/60; C07C 2601/02; C09K 2211/1007; H10K 50/12; H10K 59/122

(57) ABSTRACT

Embodiments provide a method for refining an organic material. The method for refining an organic material includes a recovery process of recovering a contaminated organic material, a first refining process of separating a primary refined material from the contaminated organic material by using a first organic solvent, and a second refining process of separating a secondary refined material from the primary refined material by using a second organic solvent that is different from the first organic solvent. In the method for refining an organic material, the primary refined material and the secondary refined material may each be separated and refined through a filter filled with a filler containing at least one of diatomaceous earth, white clay, and red clay.

### CROSS-REFERENCE TO RELATED APPLICATION(S)

This application claims priority to and benefits of Korean Patent Application No. 10-2024-0071987 under 35 U.S.C. § 119, filed on May 31, 2024, in the Korean Intellectual Property Office, the entire contents of which are incorporated herein by reference.

### BACKGROUND

### 1. Technical Field

The disclosure relates a method for refining an organic material by separating and refining a dopant material from an organic material used in a light emitting element deposition process, and to an electronic device comprising an organic material that is separated and refined through the method for refining an organic material.

### 2. Description of the Related Art

Ongoing development continues on organic electroluminescence display devices and the like as image display devices. Organic electroluminescence display devices and the like are display devices that includes a so-called self-emissive light emitting element in which holes and electrons respectively injected from a first electrode and a second electrode recombine in an emission layer, so that in the emission layer, a luminescent material emits light to achieve display.

Light emitting elements include a hole transport region containing an organic material, an emission layer, and an electron transport region between a first electrode and a second electrode. Efforts are now being made to develop technologies that are designed to recover and recycle organic materials used in manufacturing the light emitting elements.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a method for refining an organic material, in which the method is capable of separating and refining a doping material from an organic material used in a light emitting element deposition process to obtain a high purity doping material.

According to an embodiment, a method for refining an organic material may include: a recovery process of recovering a contaminated organic material from a light emitting element deposition process; a first refining process; and a second refining process. The first refining process may include: mixing the contaminated organic material, which includes impurities, material A, and material B, with a first organic solvent having a pH in a range of about 3.0 to about 4.0; and filtering the mixture through a first filter to separate a primary refined material, which includes the impurities and material B, from the contaminated organic material. The second refining process may include: mixing the primary refined material with a second organic solvent that is different from the first organic solvent; and filtering the mixture through a second filter to separate a secondary refined material, which includes material B, from the primary refined material. The first filter may be filled with a first filler, the second filter may be filled with a second filler, and the first filler and the second filler may each independently include at least one of diatomaceous earth, white clay, and red clay.

In an embodiment, the first organic solvent may include methylene chloride and a buffer solution.

In an embodiment, the buffer solution may include formic acid and ammonia.

In an embodiment, the second organic solvent may include acetonitrile.

In an embodiment, in the first refining process, material A may be dissolved by the first organic solvent and may pass through the first filter; and the primary refined material may be adsorbed onto the first filler.

In an embodiment, in the second refining process, the primary refined material, adsorbed onto the first filler, may be mixed with the second organic solvent; material B in the primary refined material may be dissolved by the second organic solvent; the dissolved material B may pass through the second filter and may be separated with the secondary refined material; and the impurities in the primary refined material may be adsorbed onto the second filler.

In an embodiment, the method may further include a third refining process that includes mixing the secondary refined material with a third organic solvent to dissolve residual impurities, and filtering the mixture to remove the residual impurities from the secondary refined material, wherein the residual impurities may be the impurities that remain in the secondary refined material after the second refining process.

In an embodiment, the third organic solvent may include ethyl acetate.

In an embodiment, the method may further include a fourth refining process that includes mixing the secondary refined material with the first organic solvent, and refiltering the mixture through the first filter to remove residual material A from the secondary refined material, wherein the residual material A is the material A that remains in the secondary refined material after the second refining process.

In an embodiment, the secondary refined material and the first organic solvent may be mixed at a volume ratio of about 1:5.

In an embodiment, in the fourth refining process, the secondary refined material may be adsorbed onto the first filler, and the residual material A may be dissolved in the first organic solvent and may pass through the first filter.

In an embodiment, the method may further include separating the secondary refined material from the first filler to obtain material B after the fourth refining process.

In an embodiment, the first filler and the second filler may each include diatomaceous earth.

In an embodiment, the first filler and the second filler may each include diatomaceous earth, white clay, and red clay; and in each of the first filler and the second filler, an amount of the diatomaceous earth may be greater than a total amount of the white clay and the red clay.

In an embodiment, material B may be a p-dopant material.

In an embodiment, the p-dopant material may include a cyano group-containing compound.

In an embodiment, the cyano group-containing compound may be a quinone derivative, an indacene derivative, a dibenzothiophene derivative, a dibenzofuran derivative, a dibenzoselenophene derivative, a carbazole derivative, an indolocarbazole derivative, an imidazole derivative, a pyridine derivative, a triazine derivative, a benzimidazole derivative, a 1,2-azaborine derivative, a 1,3-azaborine derivative, a 1,4-azaborine derivative, a borazine derivative, an aza derivative, a cyclopropylidene derivative, or a pyrene derivative.

In an embodiment, the cyano group-containing compound may include a compound represented by Formula 1:

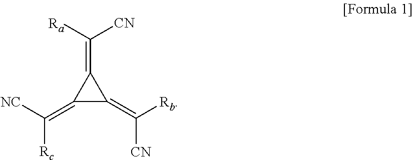

In Formula 1, Rₐ, R_{b}, and Rₑ may each independently be a cyano group, a halogen atom, a substituted or unsubstituted alkyl group having 1 to 20 carbon atoms, a substituted or unsubstituted hydrocarbon ring having 3 to 60 ring-forming carbon atoms, or a substituted or unsubstituted heterocycle having 2 to 60 ring-forming carbon atoms; and at least one of Rₐ, R_{b}, and Rₑ may each independently be a cyano group; —F; —Cl; —Br; —I; an alkyl group having 1 to 20 carbon atoms substituted with a cyano group, —F, —Cl, —Br, —I, or any combination thereof; a hydrocarbon ring having 3 to 60 ring-forming carbon atoms substituted with a cyano group, —F, —Cl, —Br, —I, or any combination thereof; or a heterocycle having 2 to 60 ring-forming carbon atoms substituted with a cyano group, —F, —Cl, —Br, —I, or any combination thereof.

In an embodiment, the cyano group-containing compound may include a compound represented by Formula A:

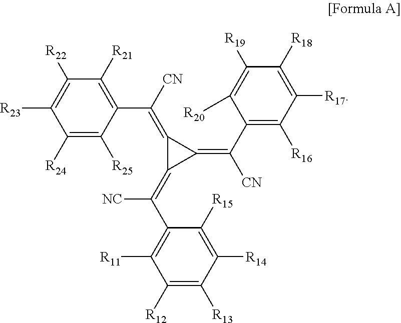

In Formula A, at least one of R₁₁ to R₂₅ may each independently be a fluorine atom; and the remainder of R₁₁ to R₂₅ may each independently be a hydrogen atom, a tritium atom, a fluorine atom, a cyano group, a substituted or unsubstituted alkyl group having 1 to 30 carbon atoms, or a substituted or unsubstituted alkenyl group having 2 to 30 carbon atoms.

In an embodiment, the first organic solvent may have a pH in a range of about 3.7 to about 3.8.

In an embodiment, the material B obtained after the second refining process may have a purity equal to or greater than about 99.5%.

According to an embodiment, a method for refining an organic material may include: recovering a contaminated organic material including impurities, material A, and material B from a light emitting element deposition process; separating and refining a primary refined material that includes impurities and material B from the contaminated organic material, by using a first organic solvent having a pH in a range of about 3.0 to about 4.0; and separating and refining a secondary refined material that includes material B from the primary refined material, by using a second organic solvent that is different from the first organic solvent.

In an embodiment, the first organic solvent may include methylene chloride; and the pH of the first organic solvent may be regulated in a range of about 3.0 to about 4.0 by a buffer solution that includes formic acid and ammonia.

According to embodiments, an electronic device may include a display device, wherein the display device may include a circuit layer disposed on a base layer, a pixel defining film disposed on the circuit layer and having pixel openings defined therein, and light emitting elements disposed on the circuit layer. At least one of the light emitting elements may include an organic material that is separated and refined through a method for refining an organic material, wherein the method may include a first refining process and a second refining process. The first refining process may include: mixing a contaminated organic material, which includes impurities, material A, and material B, with a first organic solvent having a pH in a range of about 3.0 to about 4.0; and filtering the mixture through a first filter to separate a primary refined material, which includes the impurities and material B, from the contaminated organic material. The second refining process may include: mixing the primary refined material with a second organic solvent that is different from the first organic solvent; and filtering the mixture through a second filter to separate a secondary refined material, which includes material B, from the primary refined material. The first filter may be filled with a first filler, the second filter may be filled with a second filler, and the first filler and the second filler may each independently include at least one of diatomaceous earth, white clay, and red clay.

It is to be understood that the embodiments above are described in a generic and explanatory sense only and not for the purposes of limitation, and the disclosure is not limited to the embodiments described above.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a schematic plan view of a display device according to an embodiment;

FIG. 2 is a schematic cross-sectional view of a display device according to an embodiment, showing a portion taken along virtual line I-I′ in FIG. 1;

FIG. 3 is a schematic cross-sectional view of a light emitting element according to an embodiment;

FIG. 4 is a schematic cross-sectional view of a light emitting element according to an embodiment;

FIG. 5 is a flowchart showing a method for refining an organic material according to an embodiment;

FIGS. 6A to 6C are each a schematic cross-sectional view of a filter according to an embodiment;

FIGS. 7A to 7C are each a schematic cross-sectional view that illustrate processes included in a method for refining an organic material according to an embodiment; and

FIGS. 8A and 8B are each a graph that shows the results of liquid chromatography analysis.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like reference numbers and reference characters refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may describe a group that is substituted or unsubstituted with at least one substituent selected from the group consisting of a deuterium atom, a halogen atom, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the specification, the term “bonded to an adjacent group to form a ring” may refer to a group that is bonded to an adjacent group to form a substituted or unsubstituted hydrocarbon ring or a substituted or unsubstituted heterocycle. A hydrocarbon ring may be aliphatic or aromatic. A heterocycle may be aliphatic or aromatic. A hydrocarbon ring and a heterocycle may each independently be monocyclic or polycyclic. A ring that is formed by adjacent groups being bonded to each other may itself be linked to another ring to form a spiro structure.

In the specification, the term “adjacent group” may be interpreted as a substituent that is substituted for an atom which is directly linked to an atom substituted with a corresponding substituent, as another substituent that is substituted for an atom which is substituted with a corresponding substituent, or as a substituent that is sterically positioned at the nearest position to a corresponding substituent. For example, two methyl groups in 1,2-dimethylbenzene may be interpreted as mutually “adjacent groups”, and two ethyl groups in 1,1-diethylcyclopentane may be interpreted as mutually “adjacent groups”. For example, two methyl groups in 4,5-dimethylphenanthrene may be interpreted as mutually “adjacent groups”.

In the specification, examples of a halogen atom may include a fluorine atom, a chlorine atom, a bromine atom, and an iodine atom.

In the specification, an alkyl group may be linear or branched. The number of carbon atoms in an alkyl group may be 1 to 60, 1 to 30, 1 to 20, 1 to 10, or 1 to 6. Examples of an alkyl group may include a methyl group, an ethyl group, an n-propyl group, an isopropyl group, an n-butyl group, an s-butyl group, a t-butyl group, an i-butyl group, a 2-ethylbutyl group, a 3,3-a dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and the like, but embodiments are not limited thereto.

In the specification, a cycloalkyl group may be a cyclic alkyl group. The number of carbon atoms in a cycloalkyl group may be 3 to 60, 3 to 30, 3 to 20, or 3 to 10. Examples of a cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and the like, but embodiments are not limited thereto.

In the specification, an alkenyl group may be a hydrocarbon group that includes at least one carbon-carbon double bond in the middle or at a terminus of an alkyl group having 2 or more carbon atoms. An alkenyl group may be linear or branched. The number of carbon atoms in an alkenyl group is not particularly limited, and may be 2 to 60, 2 to 30, 2 to 20 or 2 to 10. Examples of an alkenyl group may include a vinyl group, a 1-butenyl group, a 1-pentenyl group, a 1,3-butadienyl aryl group, a styrenyl group, a styryl vinyl group, and the like, but embodiments are not limited thereto.

In the specification, an alkynyl group may be a hydrocarbon group that includes at least one carbon-carbon triple bond in the middle or at a terminus of an alkyl group having 2 or more carbon atoms. An alkynyl group may be linear or branched. The number of carbon atoms in an alkynyl group is not particularly limited, and may be 2 to 30, 2 to 20, or 2 to 10. Examples of an alkynyl group may include an ethynyl group, a propynyl group, and the like, but embodiments are not limited thereto.

In the specification, a hydrocarbon ring group may be any functional group or substituent derived from an aliphatic hydrocarbon ring or an aromatic hydrocarbon ring. The number of ring-forming carbon atoms in a hydrocarbon ring group may be 3 to 60, 5 to 30, or 5 to 20. For example, a hydrocarbon ring group may be a saturated hydrocarbon ring group having 5 to 20 ring-forming carbon atoms.

In the specification, an aryl group may be any functional group or substituent derived from an aromatic hydrocarbon ring. An aryl group may be monocyclic or polycyclic. The number of ring-forming carbon atoms in an aryl group may be 6 to 60, 6 to 30, 6 to 20, or 6 to 15. Examples of an aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and the like, but embodiments are not limited thereto.

In the specification, a fluorenyl group may be substituted, and two substituents may be bonded to each other to form a spiro structure. Examples of a substituted fluorenyl group may include the groups shown below. However, embodiments are not limited thereto.

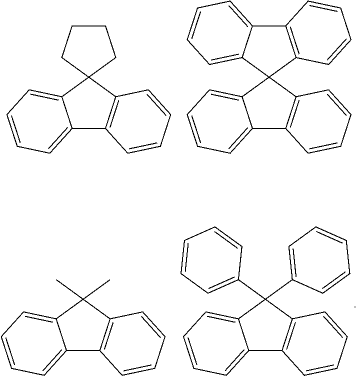

In the specification, a heterocyclic group may be any functional group or substituent derived from a ring containing at least one of B, O, N, P, S, Si, and Se as a heteroatom. A heterocyclic group may be aliphatic or aromatic. An aromatic heterocyclic group may be a heteroaryl group. An aliphatic heterocycle and an aromatic heterocycle may each independently be monocyclic or polycyclic.

When a heterocyclic group contains two or more heteroatoms, the two or more heteroatoms may be the same as or different from each other. The number of ring-forming carbon atoms in a heterocyclic group may be 2 to 60, 2 to 30, 2 to 20, or 2 to 10.

Examples of an aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and the like, but embodiments are not limited to thereto

Examples of a heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and the like, but embodiments are not limited thereto.

In the specification, the above description of an aryl group may be applied to an arylene group, except that an arylene group is a divalent group. In the specification, the above description of a heteroaryl group may be applied to a heteroarylene group, except that a heteroarylene group is a divalent group.

In the specification, a silyl group may be an alkyl silyl group or an aryl silyl group. Examples of a silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but embodiments are not limited thereto.

In the specification, the number of carbon atoms in a carbonyl group is not particularly limited, and may be 1 to 40, 1 to 30, or 1 to 20. For example, a carbonyl group may include one of the following structures, but embodiments are not limited thereto.

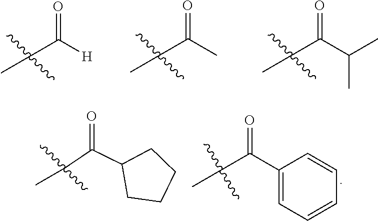

In the specification, the number of carbon atoms in a sulfinyl group or a sulfonyl group is not particularly limited, and may be 1 to 30. A sulfinyl group may be an alkyl sulfinyl group or an aryl sulfinyl group. A sulfonyl group may be an alkyl sulfonyl group or an aryl sulfonyl group.

In the specification, a thio group may be an alkyl thio group or an aryl thio group. A thio group may be a sulfur atom that is bonded to an alkyl group or to an aryl group as defined above. Examples of a thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, and the like, but embodiments are not limited to thereto.

In the specification, an oxy group may be an oxygen atom that is bonded to an alkyl group or to an aryl group as defined above. An oxy group may be an alkoxy group or an aryl oxy group. An alkoxy group may be linear, branched, or cyclic. The number of carbon atoms in an alkoxy group is not particularly limited, and may be, for example, 1 to 20, or 1 to 10.

Examples of an oxy group include a methoxy group, an ethoxy group, an n-propoxy group, an isopropoxy group, a butoxy group, a pentyloxy group, a hexyloxy group, an octyloxy group, a nonyloxy group, a decyloxy group, a benzyloxy group, and the like, but embodiments are not limited thereto.

In the specification, a boron group may be a boron atom that is bonded to an alkyl group or to an aryl group as defined above. A boron group may be an alkyl boron group or an aryl boron group. Examples of a boron group may include a dimethyl boron group, a diethyl boron group, a t-butylmethyl boron group, a diphenyl boron group, a phenyl boron group, and the like, but embodiments are not limited thereto.

In the specification, the number of carbon atoms in an amine group is not particularly limited, and may be 1 to 30. An amine group may be an alkyl amine group or an aryl amine group. Examples of an amine group may include a methylamine group, a dimethylamine group, a phenylamine group, a diphenylamine group, a naphthylamine group, a 9-methyl-anthracenylamine group, and the like, but embodiments are not limited thereto.

In the specification, an alkyl group within an alkylthio group, an alkyl sulfoxy group, an alkylaryl group, an alkylamine group, an alkyl boron group, an alkyl silyl group, or an alkyl amine group may be the same as an example of an alkyl group as described above.

In the specification, an aryl group within an aryloxy group, an arylthio group, an aryl sulfoxy group, an arylamine group, an aryl boron group, an aryl silyl group, or an aryl amine group may be the same as an example of an aryl group as described above.

In the specification, a direct linkage may be to a single bond.

In the description, the symbols

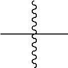

and —* each represent a bond to a neighboring atom in a corresponding formula or moiety.

Hereinafter, embodiments of the disclosure will be described with reference to the accompanying drawings.

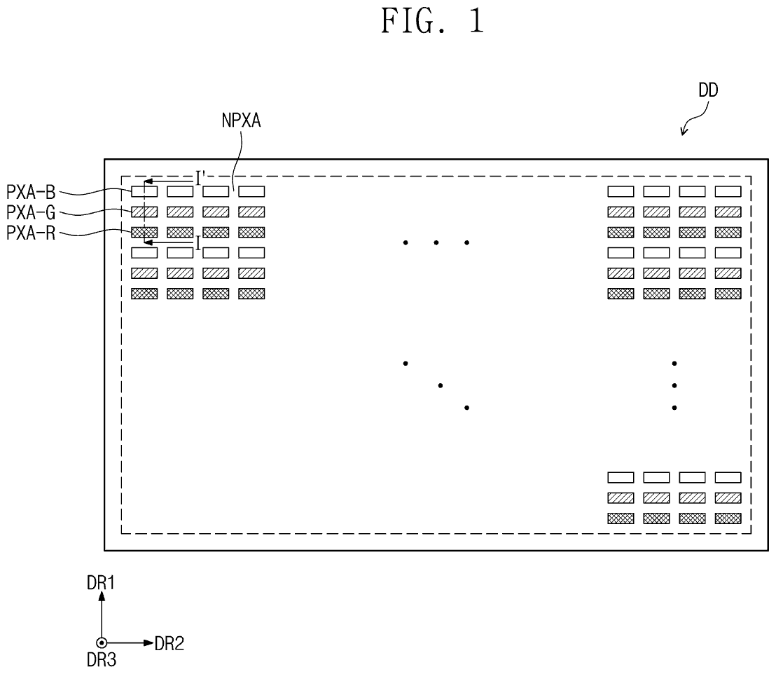

FIG. 1 is a schematic plan view of a display device DD according to an embodiment.

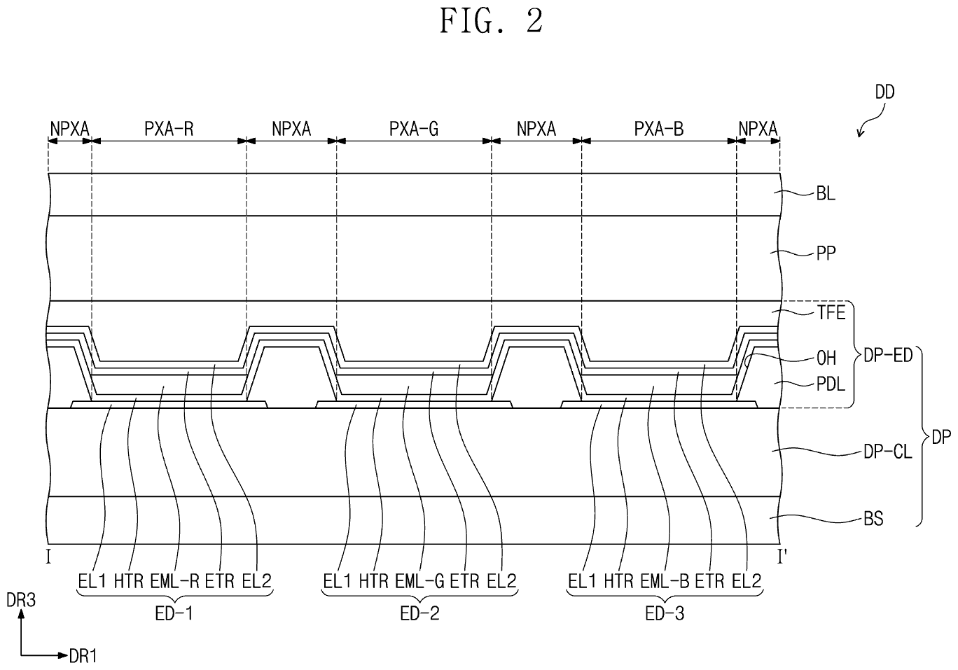

FIG. 2 is a schematic cross-sectional view of a display device DD according to an embodiment.

FIG. 2 is a schematic cross-sectional view of a portion corresponding to virtual line I-I′ in FIG. 1.

A display device DD may include a display panel DP and an optical layer PP disposed on the display panel DP. The display panel DP includes light emitting elements ED-1, ED-2, and ED-3. The display device DD may include multiples of each of the light emitting elements ED-1, ED-2, and ED-3. The optical layer PP may be disposed on the display panel DP to control light that is reflected at the display panel DP from an external light. The optical layer PP may include, for example, a polarizing layer or a color filter layer. Although not shown in the drawings, in an embodiment, the optical layer PP may be omitted from the display device DD.

A base substrate BL may be disposed on the optical layer PP. The base substrate BL may provide a base surface on which the optical layer PP is disposed. The base substrate BL may be a glass substrate, a metal substrate, a plastic substrate, or the like. However, embodiments are not limited thereto, and the base substrate BL may include an inorganic layer, an organic layer, or a composite material layer. Although not shown in the drawings, in an embodiment, the base substrate BL may be omitted.

The display device DD according to an embodiment may further include a filling layer (not shown). The filling layer (not shown) may be disposed between a display element layer DP-ED and the base substrate BL. The filling layer (not shown) may be an organic material layer. The filling layer (not shown) may include at least one of an acrylic resin, a silicone-based resin, and an epoxy-based resin.

The display panel DP may include a base layer BS, a circuit layer DP-CL provided on the base layer BS, and a display element layer DP-ED. The display element layer DP-ED may include pixel defining films PDL, light emitting elements ED-1, ED-2, and ED-3 disposed between portions of the pixel defining films PDL, and an encapsulation layer TFE disposed on the light emitting elements ED-1, ED-2, and ED-3.

The base layer BS may provide a base surface on which the display element layer DP-ED is disposed. The base layer BS may be a glass substrate, a metal substrate, a plastic substrate, or the like. However, embodiments are not limited thereto, and the base layer BS may include an inorganic layer, an organic layer, or a composite material layer.

In an embodiment, the circuit layer DP-CL may be disposed on the base layer BS, and the circuit layer DP-CL may include transistors (not shown). The transistors (not shown) may each include a control electrode, an input electrode, and an output electrode. For example, the circuit layer DP-CL may include a switching transistor and a driving transistor for driving the light emitting elements ED-1, ED-2, and ED-3 of the display element layer DP-ED.

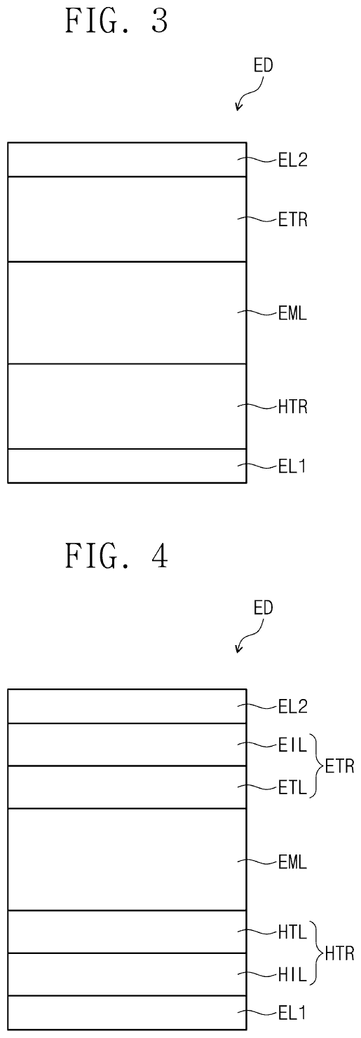

The light emitting elements ED-1, ED-2, and ED-3 may each have a structure of a light emitting element ED according to an embodiment as shown in any of FIGS. 3 and 4, which will be described later. The light emitting elements ED-1, ED-2, and ED-3 may each include a first electrode EL1, a hole transport region HTR, emission layers EML-R, EML-G, and EML-B, an electron transport region ETR, and a second electrode EL2. A method of refining a light emitting material according to an embodiment may refine a doping material used in at least one of the hole transport region HTR, the emission layers EML-R, EML-G, and EML-B, and the electron transport region ETR.

FIG. 2 shows an embodiment in which the emission layers EML-R, EML-G, and EML-B of the light emitting elements ED-1, ED-2, and ED-3 are disposed in openings OH defined in the pixel defining films PDL, and the hole transport region HTR, the electron transport region ETR, and the second electrode EL2 are each provided as a common layer that extends throughout the light emitting elements ED-1, ED-2, and ED-3. However, embodiments are not limited thereto. Although not shown in FIG. 2, in an embodiment, the hole transport region HTR and the electron transport region ETR may each be provided by being patterned in the openings OH defined in the pixel defining films PDL. For example, in an embodiment, the hole transport region HTR, the emission layers EML-R, EML-G, and EML-B, and the electron transport region ETR, and the like of the light emitting elements ED-1, ED-2, and ED-3 may be provided by being patterned through an inkjet printing method.

The encapsulation layer TFE may cover the light emitting elements ED-1, ED-2, and ED-3. The encapsulation layer TFE may seal the display element layer DP-ED. The encapsulation layer TFE may be a thin film encapsulation layer. The encapsulation layer TFE may be a single layer or a stack of multiple layers. The encapsulation layer TFE may include at least one insulating layer. The encapsulation layer TFE according to an embodiment may include at least one inorganic film (hereinafter, an encapsulation inorganic film). The encapsulation layer TFE according to an embodiment may include at least one organic film (hereinafter, an encapsulation organic film) and at least one encapsulation inorganic film.

The encapsulation inorganic film protects the display element layer DP-ED from moisture and/or oxygen, and the encapsulation organic film protects the display element layer DP-ED from foreign substances such as dust particles. The encapsulation inorganic film may include silicon nitride, silicon oxy nitride, silicon oxide, titanium oxide, aluminum oxide, or the like, but embodiments are not limited thereto. The encapsulation organic film may include an acrylic-based compound, an epoxy-based compound, or the like. The encapsulation organic film may include a photopolymerizable organic material, but embodiments are not limited thereto.

The encapsulation layer TFE may be disposed on the second electrode EL2, and may be disposed to fill the openings OH.

Referring to FIGS. 1 and 2, the display device DD may include non-light emitting regions NPXA and light emitting regions PXA-R, PXA-G, and PXA-B. The light emitting regions PXA-R, PXA-G, and PXA-B may each be a region that emits light respectively generated by the light emitting elements ED-1, ED-2, and ED-3. The light emitting regions PXA-R, PXA-G, and PXA-B may be spaced apart from each other in a plan view.

The light emitting regions PXA-R, PXA-G, and PXA-B may be regions that are separated from each other by the pixel defining films PDL. The non-light emitting regions NPXA may be regions between neighboring light emitting regions PXA-R, PXA-G, and PXA-B, and which may correspond to the pixel defining film PDL. In an embodiment, the light emitting regions PXA-R, PXA-G, and PXA-B may each correspond to a pixel. The pixel defining film PDL may separate the light emitting elements ED-1, ED-2, and ED-3. The emission layers EML-R, EML-G, and EML-B of the light emitting elements ED-1, ED-2, and ED-3 may be disposed in openings OH defined by the pixel defining films PDL and separated from each other.

The light emitting regions PXA-R, PXA-G, and PXA-B may be arranged into groups according to the color of light generated from the light emitting elements ED-1, ED-2, and ED-3. In the display device DD according to an embodiment shown in FIGS. 1 and 2, three light emitting regions PXA-R, PXA-G, and PXA-B, which respectively emit red light, green light, and blue light, are shown as an example. For example, the display device DD may include a red light emitting region PXA-R, a green light emitting region PXA-G, and a blue light emitting region PXA-B, which are distinct from one another.

In the display device DD according to an embodiment, the light emitting elements ED-1, ED-2, and ED-3 may emit light having wavelength ranges that are different from each other.

For example, in an embodiment, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may respectively correspond to the first light emitting element ED-1, the second light emitting element ED-2, and the third light emitting element ED-3.

However, embodiments are not limited thereto, and the first to third light emitting elements ED-1, ED-2, and ED-3 may emit light in a same wavelength range, or may emit light in at least one different wavelength range. For example, the first to third light emitting elements ED-1, ED-2, and ED-3 may each emit blue light.

The light emitting regions PXA-R, PXA-G, and PXA-B in the display device DD according to an embodiment may be arranged in a stripe configuration. Referring to FIG. 1, the red light emitting regions PXA-R, the green light emitting regions PXA-G, and the blue light emitting regions PXA-B may be respectively arranged along a second directional axis DR2. In another embodiment, the red light emitting region PXA-R, the green light emitting region PXA-G, and the blue light emitting region PXA-B may be arranged in this repeating order along a first directional axis DR1.

FIGS. 1 and 2 show that the light emitting regions PXA-R, PXA-G, and PXA-B are all similar in size, but embodiments are not limited thereto. In an embodiment, the light emitting regions PXA-R, PXA-G, and PXA-B may be different in size and/or shape from each other, according to a wavelength range of emitted light. The areas of the light emitting regions PXA-R, PXA-G, and PXA-B may be areas in a plan view that are defined by the first directional axis DR1 and the second directional axis DR2. A third direction axis DR3 may be perpendicular to a plane defined by the first direction axis DR1 and the second direction axis DR2.

An arrangement of the light emitting regions PXA-R, PXA-G, and PXA-B is not limited to what is shown in FIG. 1, and the order in which the red light emitting region PXA-R, the green light emitting region PXA-G, and the blue light emitting region PXA-B are arranged may be provided in various combinations, according to the display quality characteristics that are required for the display device DD. For example, the light emitting regions PXA-R, PXA-G, and PXA-B may be arranged in a pentile configuration (such as PenTile®) or in a diamond configuration (such as Diamond Pixel@).

The areas of the light emitting regions PXA-R, PXA-G, and PXA-B may be different in size from one another. For example, in an embodiment, an area of a green light emitting region PXA-G may be smaller than an area of a blue light emitting region PXA-B, but embodiments are not limited thereto.

Hereinafter, FIGS. 3 and 4 are each a schematic cross-sectional view of a light emitting element ED according to an embodiment. As shown in FIG. 3, a light emitting element ED according to an embodiment may include a first electrode EL1, a hole transport region HTR, an emission layer EML, an electron transport region ETR, and a second electrode EL2.

In comparison to FIG. 3, FIG. 4 is a schematic cross-sectional view of a light emitting element ED in which the hole transport region HTR includes a hole injection layer HIL and a hole transport layer HTL, and the electron transport region ETR includes an electron injection layer EIL and an electron transport layer ETL. Although not shown in the drawings, in embodiments, the hole transport region HTR may further include at least one of an electron blocking layer (not shown) and an auxiliary emission layer (not shown), and the electron transport region ETR may further include a hole blocking layer (not shown). For example, the hole transport region HTR may include a hole injection layer HIL, a hole transport layer HTL, and an electron blocking layer (not shown), and the electron transport region ETR may include an electron injection layer EIL, an electron transport layer ETL, and a hole blocking layer (not shown). As another example, the hole transport region HTR may include a hole injection layer HIL, a hole transport layer HTL, and an auxiliary emission layer (not shown), and the electron transport region ETR may include an electron injection layer EIL, an electron transport layer ETL, and a hole blocking layer (not shown). In embodiments, the light emitting element ED may further include a capping layer (not shown) disposed on an upper (or outer) portion of the second electrode EL2.

The first electrode EL1 has conductivity. The first electrode EL1 may be formed of a metal material, a metal alloy, or a conductive compound. The first electrode EL1 may be an anode or a cathode. However, embodiments are not limited thereto. In an embodiment, the first electrode EL1 may be a pixel electrode. The first electrode EL1 may be a transmissive electrode, a transflective electrode, or a reflective electrode. The first electrode EL1 may be a transmissive electrode, a transflective electrode, or a reflective electrode. The first electrode may include at least one of Ag, Mg, Cu, Al, Pt, Pd, Au, Ni, Nd, Ir, Cr, Li, Ca, LiF, Mo, Ti, W, In, Sn, Zn, an oxide thereof, a compound thereof, and a mixture thereof.

When the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). When the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, a compound thereof, or a mixture thereof (e.g., a mixture of Ag and Mg). In another embodiment, the first electrode EL1 may have a multilayered structure that includes a reflective film or a transflective film formed of the above-described materials, and a transparent conductive film formed of indium tin oxide (ITO), indium zinc oxide (IZO), zinc oxide (ZnO), indium tin zinc oxide (ITZO), or the like. For example, the first electrode EL1 may have a three-layered structure of ITO/Ag/ITO, but embodiments are not limited thereto. In an embodiment, the first electrode EL1 may include the above-described metal materials, a combination of at least two of the above-described metal materials, or oxides of the above-described metal materials. The first electrode EL1 may have a thickness in a range of about 700 Å to about 10,000 Å. For example, the first electrode EL1 may have a thickness in a range of about 1,000 Å to about 3,000 Å.

The hole transport region HTR may be provided on the first electrode EL1. The hole transport region HTR may include at least one of a hole injection layer HIL, a hole transport layer HTL, an auxiliary emission layer (not shown), and an electron blocking layer (not shown). The hole transport region HTR may have, for example, a thickness in a range of about 50 Å to about 15,000 Å. In the specification, the auxiliary emission layer (not shown) may also be referred to as a buffer layer.

The hole transport region HTR may have a structure consisting of a layer consisting of a single material, a structure consisting of a layer including different materials, or a structure including multiple layers including different materials.

For example, the hole transport region HTR may have a single-layered structure formed of a hole injection layer HIL or a hole transport layer HTL, or the hole transport region HTR may have a single-layered structure formed of a hole injection material or a hole transport material. For example, the hole transport region HTR may have a single-layered structure formed of different materials. In embodiments, the hole transport region HTR may have a hole injection layer HIL/hole transport layer HTL structure, a hole injection layer HIL/hole transport layer HTL/auxiliary emission layer structure, a hole injection layer HIL/auxiliary emission layer structure, a hole transport layer HTL/auxiliary emission layer structure, a hole injection layer HIL/hole transport layer HTL/auxiliary emission layer structure, or a hole injection layer HIL/hole transport layer HTL/electron blocking layer structure, in which the layers of each structure may be stacked from the first electrode EL1 in its respective stated order, but embodiments are not limited thereto.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

In the light emitting element ED according to an embodiment, the hole transport region HTR may include a compound represented by Formula H-1:

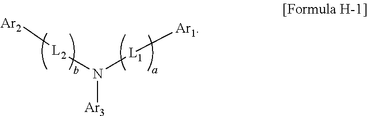

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula H-1, a and b may each independently be an integer from 0 to 10. When a or b is 2 or greater, multiple L₁ or multiple L₂ may each independently be a substituted or unsubstituted arylene group having 6 to 30 ring-forming carbon atoms or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. In Formula H-1, Ar₃ may be a substituted or unsubstituted aryl group having 6 to 30 ring-forming carbon atoms.

In an embodiment, a compound represented by Formula H-1 may be a monoamine compound. In another embodiment, a compound represented by Formula H-1 may be a diamine compound in which at least one of Ar₁, Ar₂, and Ar₃ includes an amine group as a substituent.

In an embodiment, a compound represented by Formula H-1 may be a carbazole-based compound in which at least one of Ar₁ and Ar₂ includes a substituted or unsubstituted carbazole group, or may be fluorene-based compound in which at least one of Ar₁ and Ar₂ includes a substituted or unsubstituted fluorene group.

The compound represented by Formula H-1 may be any compound selected from Compound Group H. However, the compounds listed in Compound Group H are only examples, and a compound represented by Formula H-1 is not limited to Compound Group H:

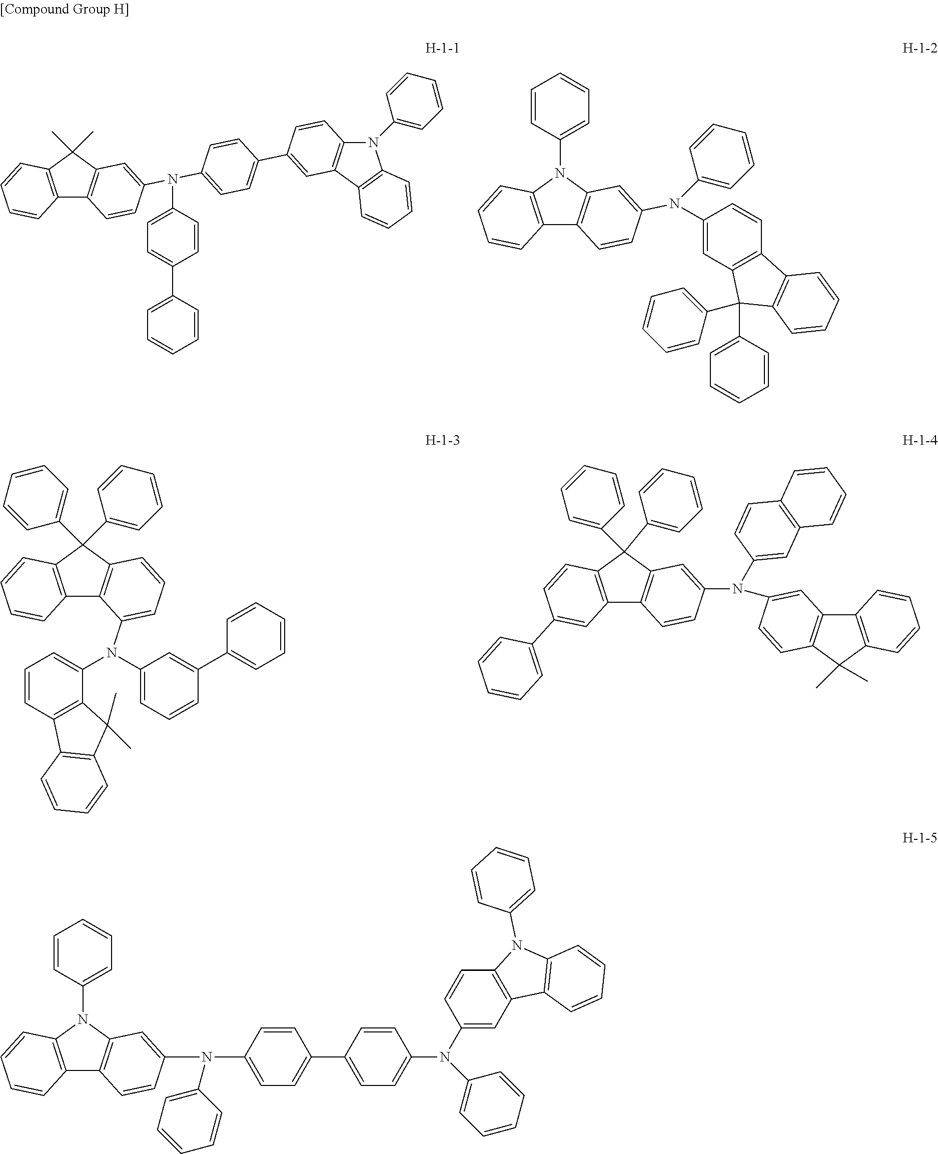 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1]

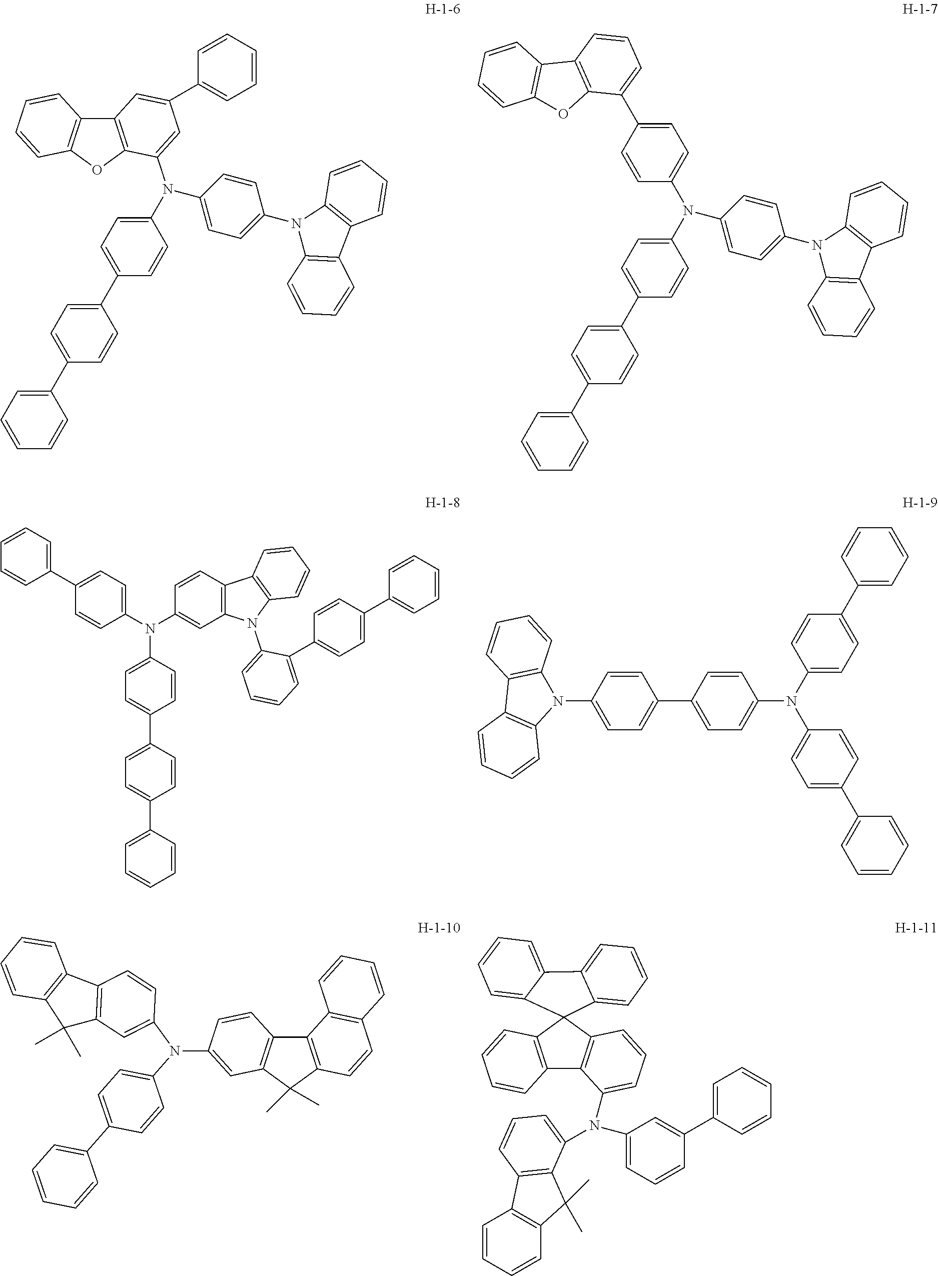

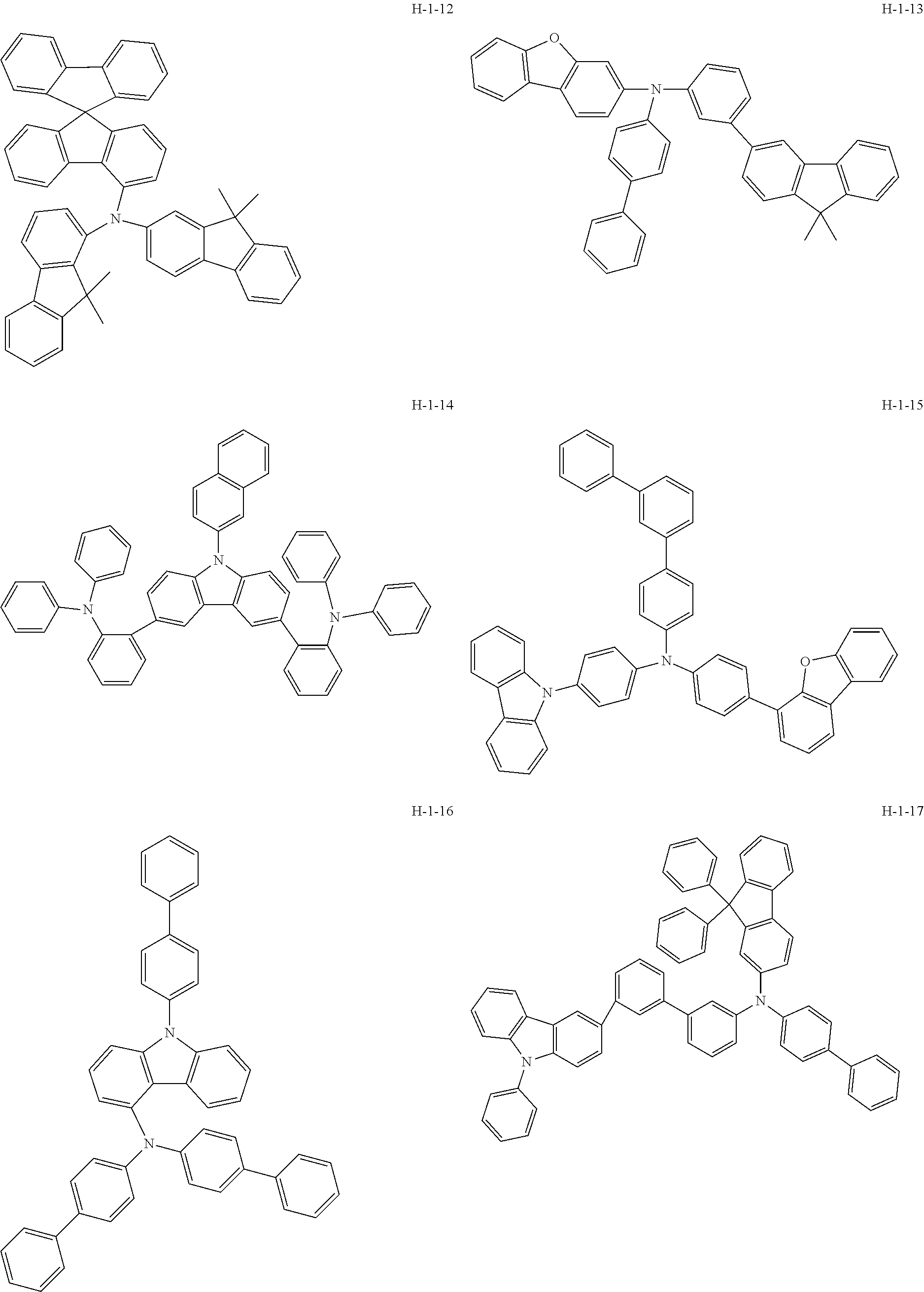

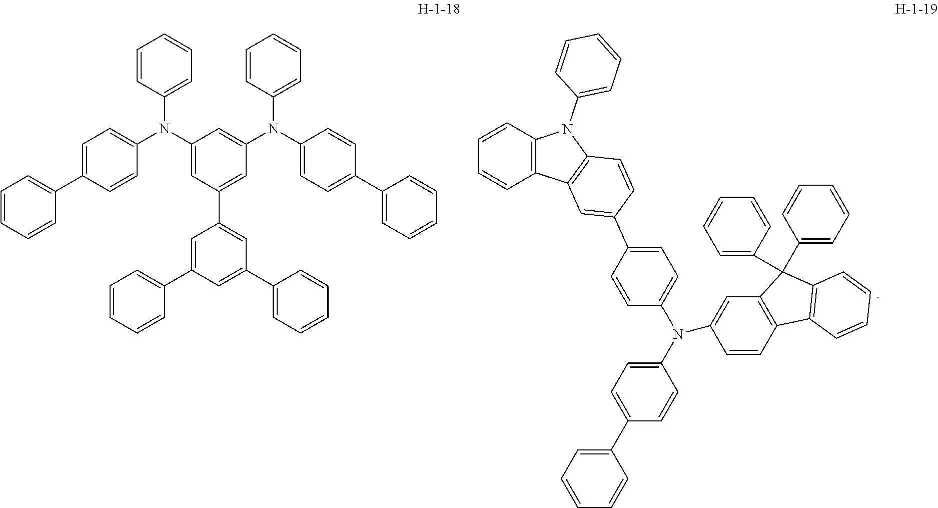

The hole transport region HTR may include a phthalocyanine compound such as copper phthalocyanine, N,N-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/Dodecylbenzenesulfonic acid (PANI/DBSA), polyaniline/camphor sulfonicacid (PANI/CSA), polyaniline/poly(4-styrenesulfonate) (PANI/PSS), N,N′-di(naphthalene-1-yl)-N,N′-diphenyl-benzidine (NPB), triphenylamine-containing polyether ketone (TPAPEK), 4-Isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HAT-CN), N-([1,1′-biphenyl]-4-yl)-9,9-dimethyl-N-(4-(9-phenyl-9H-carbazol-3-yl)phenyl)-9H-fluoren-2-amine (LHT-211), N-(9,9-diphenyl-9H-fluoren-2-yl)-N,9-diphenyl-9H-carbazol-2-amine (HT3336), or the like.

The hole transport region HTR may include a carbazole-based derivative such as N-phenyl carbazole and polyvinyl carbazole, a fluorene-based derivative, a triphenylamine-based derivative such as N,N′-bis(3-methylphenyl)-N,N′-diphenyl-[1,1-biphenyl]-4,4′-diamine (TPD), 4,4′,4′-tris(N-carbazolyl)triphenylamine (TCTA), N,N′-di(1-naphtalene-1-yl)-N,N′-diphenyl-benzidine (NPB), 4,4′-cyclohexylidene bis[N,N-bis(4-methylphenyl]benzenamine](TAPC), 4,4′-bisI[N,N′-(3-tolyl)amino]-3,3′-dimethylbiphenyl (HMTPD), 1,3-bis(N-carbazolyl)benzene (mCP), or the like.

In an embodiment, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), or the like.

The hole transport region HTR may include the compounds of the hole transport region HTR as described above in at least one of a hole injection layer HIL, a hole transport layer HTL, an auxiliary emission layer (not shown), and an electron blocking layer (not shown).

The hole transport region HTR may have a thickness in a range of about 100 Å to about 10,000 Å. For example, the hole transport region HTR may have a thickness in a range of about 100 Å to about 5,000 Å. When the hole transport region HTR includes a hole injection layer HIL, the hole injection layer HIL may have a thickness in a range of, for example, about 30 Å to about 1,000 Å. When the hole transport region HTR includes a hole transport layer HTL, the hole transport layer HTL may have a thickness in a range of about 30 Å to about 1,000 Å. When the hole transport region HTR includes an electron blocking layer, the electron blocking layer may have a thickness in a range of, for example, about 10 Å to about 1,000 Å. When the thicknesses of the hole transport region HTR, the hole injection layer HIL, the hole transport layer HTL, and the electron blocking layer satisfy the above-described ranges, satisfactory hole transport properties may be obtained without a substantial increase in driving voltage.

The hole transport region HTR may include, in addition to the above-described materials, a charge generation material to increase conductivity. The charge generation material may be uniformly or non-uniformly dispersed in the hole transport region HTR. The charge generation material may be, for example, a p-dopant. The p-dopant may be a compound that includes at least one of a cyano group, a fluorine atom, and an aromatic hydrocarbon ring in the molecular structure thereof. In an embodiment, the p-dopant may be a compound that includes at least one of a cyano group and an aromatic hydrocarbon ring in the molecular structure thereof. The p-dopant may include: a metal halide; a metal oxide; a cyano group-containing compound such as a quinone derivative, an indacene derivative, a dibenzothiophene derivative, a dibenzofuran derivative, a dibenzoselenophene derivative, a carbazole derivative, an indolocarbazole derivative, an imidazole derivative, a pyridine derivative, a triazine derivative, a benzimidazole derivative, a 1,2-azaborine derivative, a 1,3-azaborine derivative, a 1,4-azaborine derivative, a borazine derivative, an aza derivative, a cyclopropylidene derivative, a cyclopropane derivative, a pyrene derivative, and the like; or any combination thereof, but embodiments are not limited thereto. For example, the metal halide may be CuI or RbI. The metal oxide may be a tungsten oxide, a molybdenum oxide, or the like.

Examples of a quinone derivative may include tetracyanoquinodimethane (TCNQ), 2,3,5,6-tetrafluoro-7,7,8,8-tetracyanoquinodimethane (F4-TCNQ), and the like. Examples of an indacene derivative may include the following compounds:

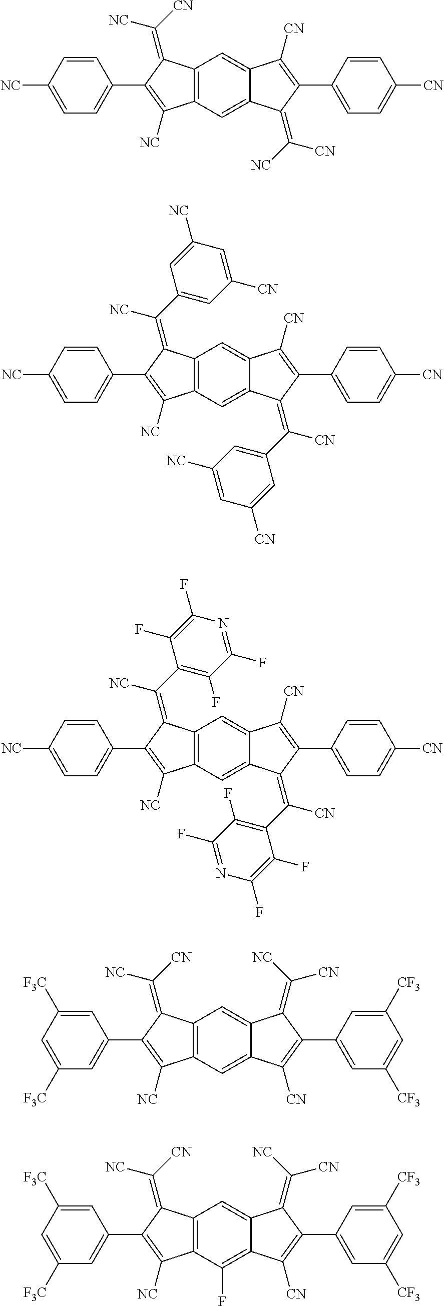

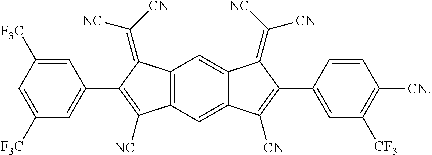 [SMILES: N#CC(C#N)=C1C(c2cc(C(F)(F)F)cc(C(F)(F)F)c2)=C(C#N)c2cc3c(cc21)C(=C(C#N)C#N)C(c1ccc(C#N)c(C(F)(F)F)c1)=C3C#N]

As described above, examples of a cyano group-containing p-dopant may include a quinone derivative, an indacene derivative, a dibenzothiophene derivative, a dibenzofuran derivative, a dibenzoselenophene derivative, a carbazole derivative, an indolocarbazole derivative, an imidazole derivative, a pyridine derivative, a triazine derivatives, a benzimidazole derivative, a 1,2-azaborine derivative, a 1,3-azaborine derivative, a 1,4-azaborine derivative, a borazine derivative, an aza derivative, a cyclopropylidene derivative, a cyclopropane derivative, a pyrene derivative, and the like. Examples of a dibenzofuran derivative may include the following compounds, but embodiments are not limited thereto:

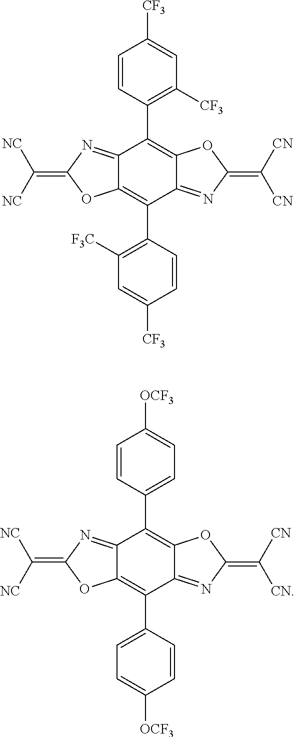 [SMILES: N#CC(C#N)=c1nc2c(-c3ccc(C(F)(F)F)cc3C(F)(F)F)c3oc(=C(C#N)C#N)nc3c(-c3ccc(C(F)(F)F)cc3C(F)(F)F)c2o1 ; N#CC(C#N)=c1nc2c(-c3ccc(OC(F)(F)F)cc3)c3oc(=C(C#N)C#N)nc3c(-c3ccc(OC(F)(F)F)cc3)c2o1]

Another example of a cyano group-containing compound may include dipyrazino[2,3-f: 2′,3′-h]quinoxaline-2,3,6,7,10,11-hexacarbonitrile (HAT-CN), and an example of a cyclopropylidene derivative may include a compound represented by Formula 1, but embodiments are not limited thereto. In the specification, the p-dopant may be referred to as a p-dopant material.

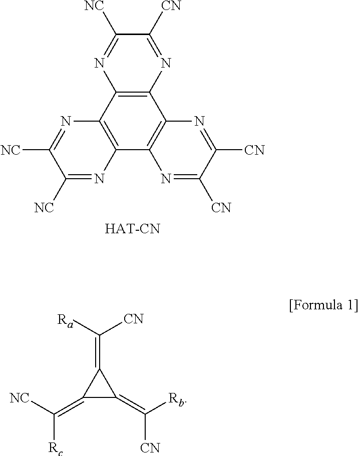

In Formula 1, Rₐ, R_{b}, and Rₑ may each independently be a cyano group, a halogen atom, a substituted or unsubstituted alkyl group having 1 to 20 carbon atoms, a substituted or unsubstituted hydrocarbon ring having 3 to 60 ring-forming carbon atoms, or a substituted or unsubstituted heterocycle having 2 to 60 ring-forming carbon atoms. For example, Rₐ, R_{b}, and Rₑ may each independently be a substituted or unsubstituted cycloalkyl group having 3 to 60 ring-forming carbon atoms, a substituted or unsubstituted aliphatic heterocyclic group having 2 to 60 ring-forming carbon atoms, a substituted or unsubstituted aryl group having 6 to 60 ring-forming carbon atoms, or a substituted or unsubstituted heteroaryl group having 2 to 60 ring-forming carbon atoms.

In Formula 1, at least one of Rₐ, R_{b}, and Rₑ may each independently be: a cyano group; —F; —Cl; —Br; —I; an alkyl group having 1 to 20 carbon atoms substituted with a cyano group, —F, —Cl, —Br, —I, or any combination thereof; a hydrocarbon ring having 3 to 60 ring-forming carbon atoms substituted with a cyano group, —F, —Cl, —Br, —I, or any combination thereof; or a heterocycle having 2 to 60 ring-forming carbon atoms substituted with a cyano group, —F, —Cl, —Br, —I, or any combination thereof.

For example, at least one of Rₐ, R_{b}, and Rₑ may each independently be a cyano group, a halogen atom, an alkyl group having 1 to 20 carbon atoms in which at least one hydrogen atom is substituted with a cyano group or a halogen atom, a cycloalkyl group having 3 to 60 ring-forming carbon atoms in which at least one hydrogen atom is substituted with a cyano group or a halogen atom, an aryl group having 5 to 60 ring-forming carbon atoms in which at least one hydrogen atom is substituted with a cyano group or a halogen atom, an aliphatic heterocyclic group having 2 to 60 ring-forming carbon atoms in which at least one hydrogen atom is substituted with a cyano group or a halogen atom, or a heteroaryl group having 2 to 60 ring-forming carbon atoms in which at least one hydrogen atom is substituted with a cyano group or a halogen atom.

In an embodiment, the cyano group-containing compound represented by Formula 1 may be represented by Formula A. In an embodiment, the p-dopant may include a compound represented by Formula A:

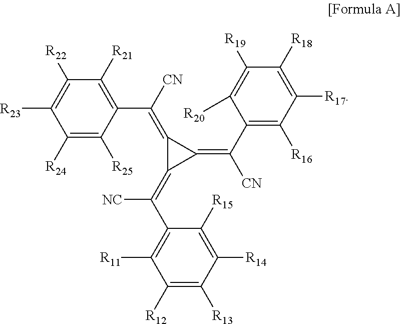

In Formula A, at least one of R₁₁ to R₂₅ may each independently be a fluorine atom (—F); and the remainder of R₁₁ to R₂₅ may each independently be a hydrogen atom, a tritium atom, a fluorine atom, a cyano group, a substituted or unsubstituted alkyl group having 1 to 30 carbon atoms, or a substituted or unsubstituted alkenyl group having 2 to 30 carbon atoms. For example, at least one of R₁₁ to R₁₅, at least one of R₁₆ to R₂₀, and at least one of R₂₁ to R₂₅ may each be a fluorine atom, and the remainder thereof may each be a cyano group, but embodiments are not limited thereto.

For example, a compound represented by Formula A may be 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP-9), which is represented by Formula A-1, but embodiments are not limited thereto.

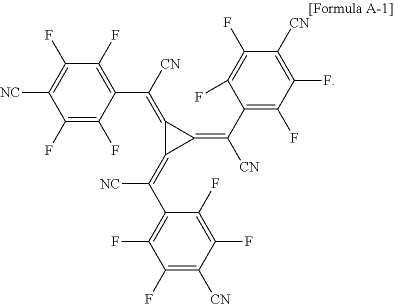 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

In an embodiment, the hole injection layer HIL may include a cyano group-containing compound as a p-dopant. For example, the hole injection layer HIL may include a compound represented by Formula 1 as a p-dopant, but embodiments are not limited thereto.

As described above, the hole transport region HTR may further include at least one of an auxiliary emission layer and an electron blocking layer, in addition to the hole injection layer HIL and the hole transport layer HTL. The auxiliary emission layer may compensate for a resonance distance according to a wavelength of light emitted from the emission layer EML and may regulate hole charge balance to increase light emission efficiency. The auxiliary emission layer may also prevent electrons from being injected into the hole transport region HTR. Materials which may be included in the hole transport region HTR may be included in the auxiliary emission layer. The electron blocking layer may prevent electrons from being injected from the electron transport region ETR to the hole transport region HTR.

The hole transport region HTR may be formed of an organic material including a doping material in a deposition process for preparing a light emitting element ED. The hole transport region HTR may include material A and material B. In the specification, material A may be a host material, and material B may be a doping material.

For example, the hole injection layer HIL may be formed of an organic material that includes a doping material and a host material. The method for refining an organic material according to an embodiment may involve recovering an organic material that is used in a deposition process of the light emitting element ED, wherein the method for refining an organic material may be used after the deposition process of the light emitting element ED, and wherein the method for refining an organic material separates and refines the doping material from the organic material. For example, the method for refining an organic material according to an embodiment may involve recovering an organic material containing a doping material used in forming a hole transport region HTR, and separating and refining the doping material from the recovered organic material. The method for refining an organic material according to an embodiment may involve recovering and recycling doping materials from organic materials that are otherwise discarded after a deposition process of the light emitting element ED, and thus may contribute to cost reduction.

Hereinafter, a method for separating and refining a doping material from an organic material used in a deposition process of the hole transport region HTR of the light emitting element ED will be described, but embodiments are not limited thereto. The emission layer EML and the electron transport region ETR included in the light emitting element ED may also be formed of an organic material containing a doping material, and the method for refining an organic material according to an embodiment may also be applied to a process for separating and refining a doping material from an organic material used in a deposition process of the emission layer EML or used in a deposition process of the electron transport region ETR.

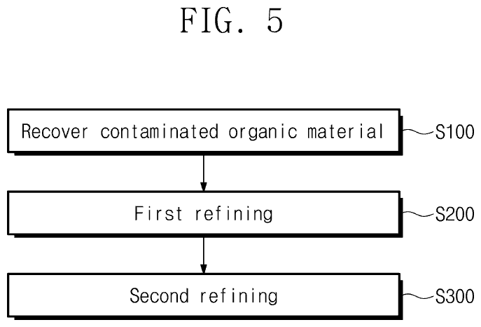

FIG. 5 is a flowchart showing a method for refining an organic material according to an embodiment. Referring to FIG. 5, the method for refining an organic material according to an embodiment may include a process of recovering a contaminated organic material S100, a first refining process S200, and a second refining process S300. In embodiments, the method for refining an organic material may further include a third refining process and/or a fourth refining process.

The process of recovering a contaminated organic material 5100 may be a process of recovering a doping material used in a deposition process of the light emitting element ED. For example, a doping material used to form a hole transport region HTR during a deposition process of the light emitting element ED may be recovered. The doping material used in the deposition process of the light emitting element ED may not evaporate after the deposition process of the light emitting element ED, and the doping material may remain in a crucible or in deposition equipment such as a deposition plate. In the deposition process of the light emitting element ED, the hole transport region HTR may be formed of an organic material containing a host material in addition to the doping material, and accordingly, after the deposition process of the light emitting element ED, the host material, in addition to the doping material, may remain in the deposition equipment and the like, and alongside that, impurities may also remain. Accordingly, when the doping material is recovered from a crucible, deposition equipment, or the like after the deposition process of the light emitting element ED, the doping material may be mixed with impurities and/or host materials. For example, the doping material used in the deposition process of the light emitting element ED may be recovered as a contaminated organic material including impurities, host materials, and doping materials from deposition equipment and the like. The impurities contained in the contaminated organic material may be impurities resulting from thermal decomposition and other materials contaminated during the deposition process. For example, the impurities may be carbon lumps formed by a material used in the deposition process of the light emitting element ED, or ash, but embodiments are not limited thereto.

In an embodiment, the contaminated organic material may be recovered by placing deposition equipment in which the contaminated organic material remains in a vacuum chamber, applying heat to sublimate the material, cooling the resulting product to an appropriate temperature or lower, and crystallizing the product. However, embodiments are not limited thereto, and the contaminated organic material may be recovered by physical means (for example, by scraping, wiping, or cleaning deposition equipment) or by otherwise removing the contaminated organic material from deposition equipment.

The method for refining an organic material according to an embodiment may involve recovering a contaminated organic material after a deposition process of the light emitting element ED, and separating and refining a doping material from the contaminated organic material to high purity. For example, the method for refining an organic material according to an embodiment may involve recovering an organic material containing a doping material used in forming a hole transport region HTR, and separating and refining a doping material from the organic material to high purity.

In the method for refining an organic material according to an embodiment, the doping material that is subjected to separation and refining may be the p-dopant as described above. The method for refining an organic material according to an embodiment may involve separating and refining a p-dopant from a contaminated organic material. The p-dopant that is separated and refined through the method for refining an organic material according to an embodiment may be a cyano group-containing compound. For example, the p-dopant may be a cyano group-containing compound containing at least one fluorine atom (—F) in the molecular structure thereof.

For example, the p-dopant that is separated and refined through the method for refining an organic material according to an embodiment may include a compound represented by Formula 1 as described above. As another example, the p-dopant may include a compound represented by Formula A as described above.

The method for refining an organic material according to an embodiment may involve separating and refining a compound represented by Formula A, such as NDP-9, which may be used as a doping material when forming a hole injection layer HIL. However, embodiments are not limited thereto, and when the p-dopant contains a cyano group, a fluorine atom, and an aromatic hydrocarbon ring in the molecular structure thereof, the p-dopant may be separated and refined to high purity from the contaminated organic material through the method for refining an organic material according to an embodiment. For example, the p-dopant separated and refined through the method for refining an organic material according to an embodiment may include a compound containing at least one cyano group, at least one fluorine atom, and a pyrene group as an aromatic hydrocarbon ring in the molecular structure thereof. For example, the p-dopant may include a pyrene moiety-containing compound. The method for refining an organic material according to an embodiment may involve separating and refining the pyrene moiety-containing compound from a contaminated organic material to high purity.

For example, the pyrene moiety-containing compound may be represented by Formula B, but embodiments are not limited thereto.

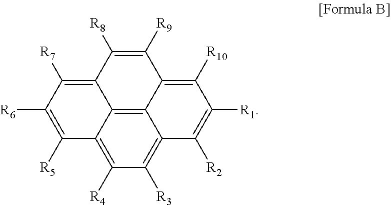

In Formula B, R₁ to R₁₀ may each independently be a tritium atom, a halogen atom, a cyano group, a substituted or unsubstituted alkyl group having 1 to 30 carbon atoms, or a substituted or unsubstituted alkenyl group having 2 to 30 carbon atoms. In an embodiment, when R₁ to R₁₀ each contains a halogen atom, the halogen atom may be a fluorine atom. In another embodiment, when R₁ to R₁₀ are each substituted, R₁ to R₁₀ may each independently be substituted with a cyano group or the like, but embodiments are not limited thereto.

In Formula B, at least one of R₁ to R₁₀ may each independently be a fluorine atom, and at least one of the remainder of R₁ to R₁₀ that are not a fluorine atom may each independently be a cyano group or an alkenyl group having 2 to 30 carbon atoms substituted with a cyano group. For example, R₁ and R₆ may each independently be an alkenyl group having 2 to 30 carbon atoms substituted with a cyano group, and R₂ to R₅ and R₇ to R₁₀ may each be a fluorine atom, but embodiments are not limited thereto.

In an embodiment, the pyrene moiety-containing compound represented by Formula B may be represented by Formula B-1. For example, the pyrene moiety-containing compound represented by Formula B-1 may be 2-(7-dicyanomethylene-1,3,4,5,6,8,9,10-octafluoro-7H-pyren-2-ylidene)-malononitrile.

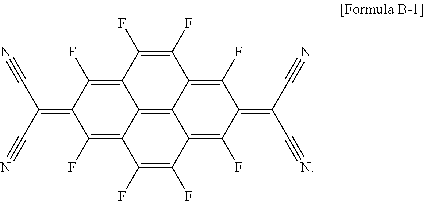 [SMILES: N#CC(C#N)=c1c(F)c2c(F)c(F)c3c(F)c(=C(C#N)C#N)c(F)c4c(F)c(F)c(c1F)c2c34]

The method for refining an organic material according to an embodiment may involve separating and refining not only NDP-9 but also a pyrene moiety-containing compound, such as 2-(7-dicyanomethylene-1,3,4,5,6,8,9,10-octafluoro-7H-pyren-2-ylidene)-malononitrile.

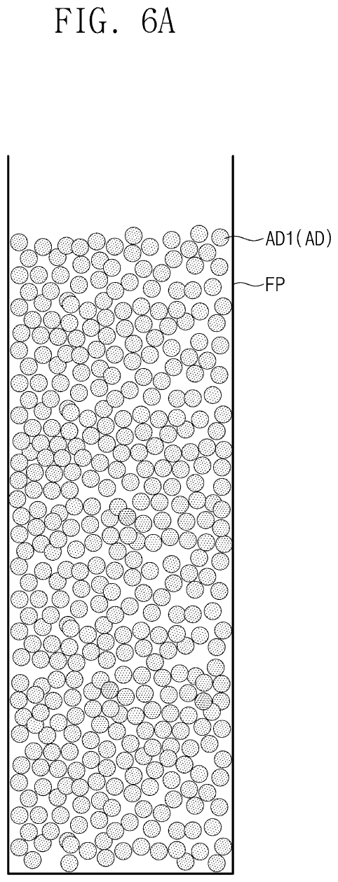

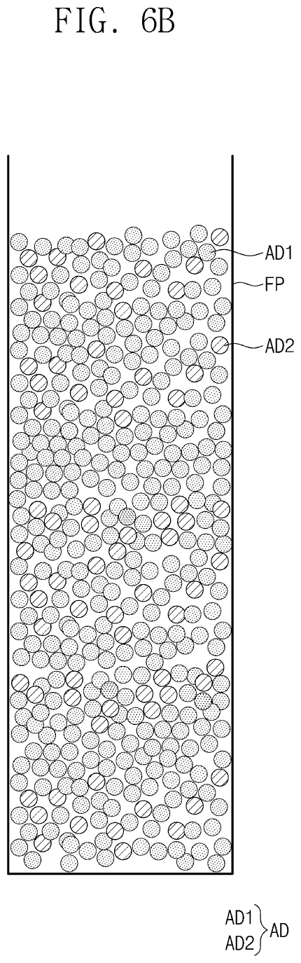

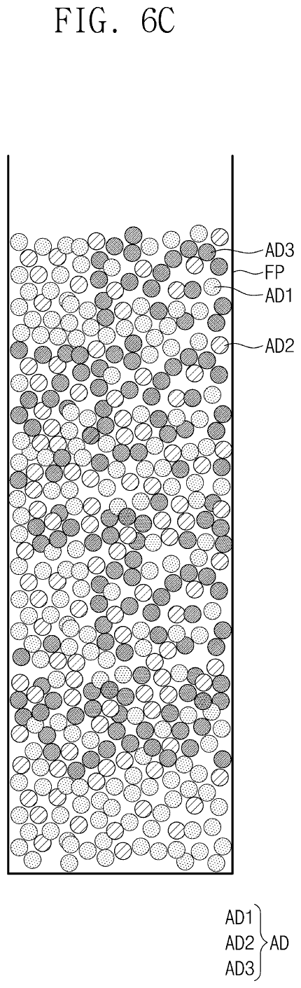

FIGS. 6A to 6C are each a schematic cross-sectional view of a filter according to an embodiment. FIGS. 6A to 6C are each different from each other at least in that a filter FP contains a different filler AD. FIG. 6A shows a filter FP that is filled with a filler AD including a first material AD1. In comparison to FIG. 6A, FIG. 6B shows a filter FP in which the filler AD further includes a second material AD2. In comparison to FIG. 6A, FIG. 6C shows a filter FP in which the filler AD further includes a second material AD2 and a third material AD3.

The filters FP shown in FIGS. 6A to 6C may each be used in the first refining process S200 and the second refining process 5300 (see FIG. 5). The method for refining an organic material according to an embodiment may involve separating a primary refined material and a secondary refined material from a contaminated organic material through the filter FP.

In an embodiment, the filter FP may be filled with the filler AD. The filler AD may include at least one of a first material AD1, a second material AD2, and a third material AD3. The first material AD1, the second material AD2, and the third material AD3 may each independently include a material selected from diatomaceous earth, white clay, and red clay.

For example, the filler AD may include at least one of diatomaceous earth, white clay, and red clay. The method for refining an organic material according to an embodiment uses a filter FP containing a filler AD that is stable when refining doping materials, is readily available, and is affordable, and may thus provide substantial cost savings. The method for refining an organic material according to an embodiment is beneficial in that the refining method is simple, as it uses a filler AD containing at least one of diatomaceous earth, white clay, and red clay, and the method allows only a doping material to be efficiently and quickly obtained from a contaminated organic material.

Referring to FIG. 6A, the filter FP may be filled with a filler AD containing the first material AD1. The first material AD1 may be diatomaceous earth, white clay, or red clay. In an embodiment, the first material AD1 may be diatomaceous earth, but embodiments are not limited thereto. For example, the first material AD1 may be white clay or red clay.

Referring to FIG. 6B, the filter FP may be filled with a filler AD including the first material AD1 and the second material AD2. The first material AD1 and the second material AD2 may each independently be diatomaceous earth, white clay, and/or red clay. The first material AD1 and the second material AD2 may be different from each other. For example, the first material AD1 may be diatomaceous earth, and the second material AD2 may be white clay or red clay.

In an embodiment, when the filter FP is filled with the first material AD1 and the second material AD2, an amount of the first material AD1 may be greater than an amount of the second material AD2. For example, when the filler AD in the filter FP includes diatomaceous earth as the first material AD1 and white clay or red clay as the second material AD2, an amount of diatomaceous earth in the filter FP may be greater than an amount of white clay or red clay.

Referring to FIG. 6C, the filter FP may be filled with a filler AD including the first material AD1, the second material AD2, and the third material AD3. The first material AD1, the second material AD2, and the third material AD3 may each independently be diatomaceous earth, white clay, and/or red clay. The first to third materials AD1 to AD3 may be different from each other. For example, the first material AD1 may be diatomaceous earth, the second material AD2 may be white clay, and the third material AD3 may be red clay.

In an embodiment, when the filter FP is filled with the first material AD1, the second material AD2, and the third material AD3, an amount of the first material AD1 may be greater than an amount of the second material AD2 and an amount of the third material AD3. In an embodiment, an amount of the first material AD1 in the filter FP may be greater than a total amount of the second material AD2 and the third material AD3. For example, the filler AD in the filter FP may include diatomaceous earth as the first material AD1, white clay as the second material AD2, and red clay as the third material AD3, and in the filter FP, an amount of diatomaceous earth may be greater than a total amount of white clay and red clay.

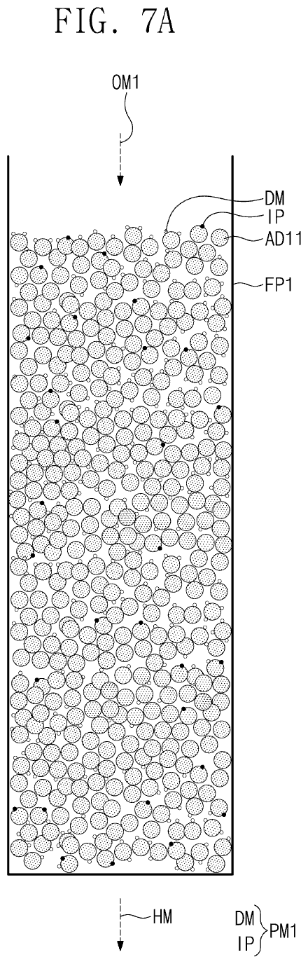

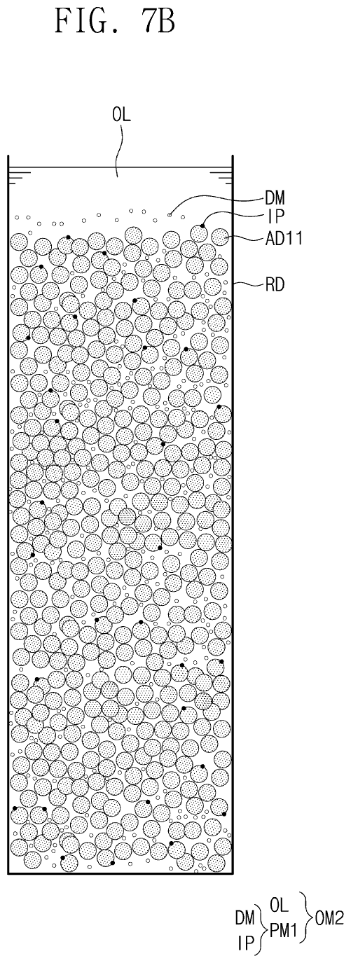

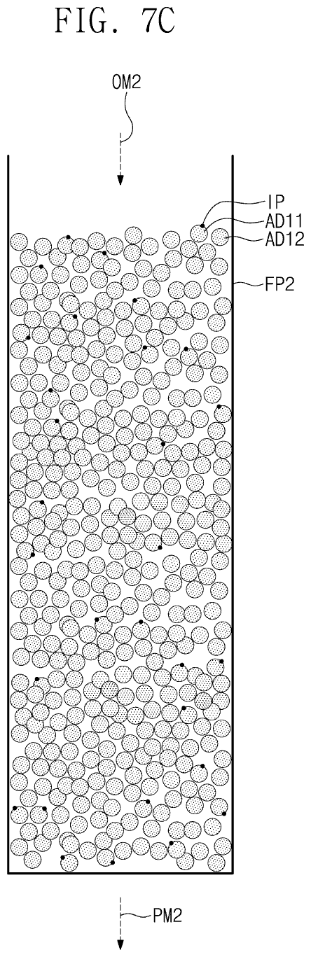

FIGS. 7A to 7C are each a schematic cross-sectional view that illustrate processes included in a method for refining an organic material according to an embodiment. FIG. 7A is a view that describes the first refining process 5200, and FIGS. 7B and 7C are each a view that describes the second refining process 5300. FIGS. 7A and 7C respectively show a first filter FP1 and a second filter FP2, each corresponding to the filter FP of FIG. 6A as an example, but embodiments are not limited thereto. The first refining process 5200 and the second refining process 5300 may each independently use the filter FP of FIG. 6B or FIG. 6C.

Referring to FIGS. 5 and 7A, in the first refining process S200, a host material HM included in a contaminated organic material may be removed using the first filter FP1. For example, the host material HM removed in the first refining process S200 may be N-([1,1′-biphenyl]-4-yl)-9,9-dimethyl-N-(4-(9-phenyl-9H-carbazol-3-yl)phenyl)-9H-fluoren-2-amine (LHT-211), N-(9,9-diphenyl-9H-fluoren-2-yl)-N,9-diphenyl-9H-carbazol-2-amine (HT3336), or the like, but embodiments are not limited thereto. In the first refining process S200, a primary refined material PM1 containing impurities IP and a doping material DM may be separated from a contaminated organic material. In the method for refining an organic material according to an embodiment, the first refining process 5200 may be performed at least once.

The first filter FP1 used in the first refining process 5200 may be filled with a first filler AD11. The first filler AD11 may correspond to the filler AD described above with reference to any of FIGS. 6A to 6C. The contents of the first filler AD11 may be the same as the contents of the filler AD according to one of FIGS. 6A to 6C. For example, the first filler AD11 may include at least one of diatomaceous earth, white clay, and red clay. For example, the first filler AD11 may include diatomaceous earth, but embodiments are not limited thereto.

According to an embodiment, in the first refining process S200, a contaminated organic material may be mixed with a first organic solvent. The contaminated organic material and the first organic solvent may be mixed at a volume ratio of about 1:5 (contaminated organic material:first organic solvent), but embodiments are not limited thereto. The contaminated organic material and the first organic solvent may be mixed to form a first mixture OM1. In the first refining process S200, when the first mixture OM1 is filtered through the first filter FP1, the primary refined material PM1 may be filtered by the first filler AD11 in the first filter FP1, and the host material HM may pass through the first filter FP1. Although not shown in the drawings, in an embodiment, when the host material HM is discharged below the first filter FP1, the first organic solvent may also pass through the first filter FP1 along with the host material HM. The primary refined material PM1 filtered be the first filter FP1 may include the impurities IP and the doping material DM.

The first organic solvent may be an organic solvent wherein the pH is regulated. In the method for refining an organic material according to an embodiment, the pH of the first organic solvent may be adjusted and used within a specific range, and the host material HM may thus be dissolved without dissolving the doping material DM in the first organic solvent. The host material HM discharged from the first filter FP1 may be discharged in the form of being dissolved in the first organic solvent. The host material HM dissolved by the first organic solvent may pass through the first filter FP1 and may be removed from the contaminated organic material. In embodiments, at least some of the impurities IP may be dissolved by the first organic solvent. Accordingly, different from what is shown in FIG. 7A, at least some of the impurities IP dissolved in the first organic solvent may pass through the first filter FP1 and may be discharged below the first filter FP1, and thus be removed from the contaminated organic material.

According to embodiments, the first organic solvent may have a pH in a range of about 3.0 to about 4.0. In an embodiment, the first organic solvent may have a pH in a range of about 3.7 to about 3.8. For example, the first organic solvent may have a pH of about 3.75, but embodiments are not limited thereto. When the pH of the first organic solvent is less than about 3.0, the doping material DM and host material HM in the contaminated organic material may both dissolve and be discharged below the first filter FP1. When the pH of the first organic solvent is greater than about 4.0, the host material HM may have reduced solubility and may thus not be fully dissolved in the first organic solvent. Accordingly, when the pH of the first organic solvent is greater than about 4.0, the host material HM may also be filtered by the first filter FP1, and may thus not be sufficiently removed from the contaminated organic material.

The first organic solvent may include at least one organic solvent. In an embodiment, the first organic solvent may include methylene chloride and a buffer solution. In an embodiment, the buffer solution may include formic acid and ammonia. Methylene chloride may be included in the first organic solvent as a means to dissolve the host material HM. Formic acid and ammonia may adjust and regulate the pH of the first organic solvent. The inclusion of methylene chloride in the first organic solvent in combination with the inclusion of formic acid and ammonia in the buffer solution may allow the adjustment and regulation of pH in a range of about 3.0 to about 4.0. For example, the first organic solvent may be prepared by dissolving 0.943 mL of formic acid in water in a 250 mL volumetric flask, adding aqueous ammonia to prepare 100 mL of a buffer solution having a pH in a range of about 3.7 to about 3.8, and mixing the buffer solution with methylene chloride, but embodiments are not limited thereto.

In comparison to an organic solvent that contains only methylene chloride, the first organic solvent according to an embodiment includes methylene chloride, formic acid, and ammonia. Thus, the first organic solvent has a pH that is adjusted and regulated within a range that prevents the doping material DM from dissolving and that selectively dissolves the host material HM and at least some of the impurities IP. Accordingly, when the first mixture OM1 is filtered through the first filter FP1, a primary refined material PM1, in which the host material HM is removed from the contaminated organic material, may be obtained.

In the first refining process 5200, the filtering of the first mixture OM1 may be performed (and may be repeated) until the host material HM is not discharged from the first filter FP1. Thin layer chromatography (TLC) may be used to determine whether the host material HM is discharged from the first filter FP1. By using TLC at specified time intervals to determine whether the host material HM is discharged from the first filter FP1, the first mixture OM1 may be filtered accordingly until the discharge of the host material HM is complete. For example, whether the host material HM is discharged from the first filter FP1 may be determined by performing TLC at intervals of about 1 to 5 minutes, and thus, it may be determined whether to continue or stop filtration of the first mixture OM1 using the first filter FP1. In determining the presence or absence of the host material HM discharged from the first filter FP1 through TLC, when the host material HM is no longer detected, it may be determined that the host material HM is completely removed from the contaminated organic material and thus filtration of the first mixture OM1 may be stopped.

In an embodiment, the impurities IP and the doping material DM that are not dissolved by the first organic solvent may be adsorbed onto the first filler AD11. The impurities IP and doping material DM adsorbed onto the first filler AD11 may be the primary refined material PM1 in which the host material HM is removed from the contaminated organic material. The primary refined material PM1, adsorbed onto the first filler AD11, may be separated from the contaminated organic material.

Referring to FIGS. 5, 7B, and 7C, in the second refining process 5300, the impurities IP may be removed from the primary refined material PM1, using the second filter FP2. In the second refining process 5300, a secondary refined material PM2 containing the doping material DM may be separated from the primary refined material PM1. The contaminated organic material, following the first refining process S200 and the second refining process 5300, is free of the host material HM and the impurities IP, and may thus be refined into the secondary refined material PM2. The secondary refined material PM2 may be substantially free of the host material HM (see FIG. 7A) and substantially free of the impurities IP. In the method for refining an organic material according to an embodiment, the second refining process S300 may be performed at least once.

Referring to FIGS. 5 and 7B, in the second refining process S300, the primary refined material PM1 may be mixed with a second organic solvent OL. The second organic solvent OL may be mixed with the primary refined material PM1 at a volume ratio of about 50:1 (second organic solvent: primary refined material), but embodiments are not limited thereto. The primary refined material PM1 and the second organic solvent OL may be mixed to form a second mixture OM2.

In the second refining process S300, the primary refined material PM1, adsorbed onto the first filler AD11, may be mixed with the second organic solvent OL. For example, after the first refining process S200, the first filler AD11 onto which the primary refined material PM1 is adsorbed may be placed in a reactor RD, and the second organic solvent OL may be added thereto and stirred. Accordingly, the primary refined material PM1 and the second organic solvent OL may form the second mixture OM2.

The method for refining an organic material according to an embodiment may desorb the doping material DM from the first filler AD11 by mixing the primary refined material PM1 and the second organic solvent OL in the second refining process S300. For example, as shown in FIG. 7B, when the primary refined material PM1 adsorbed onto the first filler AD11 and the second organic solvent OL are mixed, the second organic solvent OL dissolves the doping material DM included in the primary refined material PM1, and accordingly, the doping material DM may be desorbed from the first filler AD11.

The mixing of the primary refined material PM1 with the second organic solvent OL may allow the impurities IP, the doping material DM, and the second organic solvent OL to form the second mixture OM2. Thus, the second mixture OM2 may include the primary refined material PM1 and the second organic solvent OL. In the second mixture OM2, the impurities IP may remain adsorbed onto the first filler AD11, and the doping material DM may be desorbed from the first filler AD11.

In an embodiment, the second organic solvent OL may include acetonitrile. Acetonitrile may dissolve the doping material DM contained in the primary refined material PM1. Accordingly, when the second organic solvent OL containing acetonitrile and the primary refined material PM1 are mixed after the first refining process S200, the doping material DM may be desorbed from the first filler AD11.

Referring to FIGS. 7B and 7C, the second mixture OM2 may be filtered through the second filter FP2 to obtain the secondary refined material PM2. When the second mixture OM2 is filtered through the second filter FP2, the impurities IP contained in the second mixture OM2 may be filtered by a second filler AD12 in the second filter FP2, and the secondary refined material PM2 may pass through the second filter FP2. The secondary refined material PM2, which passed through the second filter FP2, may include the doping material DM.

In the second refining process 5300, the doping material DM is dissolved in the second organic solvent OL, and may thus pass through the second filter FP2 without being adsorbed onto the second filler AD12. Accordingly, in the second refining process 5300, the secondary refined material PM2 containing the doping material DM, which is separated from the primary refined material PM1, may be obtained. This secondary refined material PM2 may include the second organic solvent OL, which passed through the second filter FP2, together with the doping material DM.

In the second refining process 5300, the impurities IP may not be dissolved in the second organic solvent OL. The impurities IP may remain adsorbed onto the first filler AD11 in the second mixture OM2. Accordingly, when the second mixture OM2 is filtered through the second filter FP2, the impurities IP adsorbed onto the first filler AD11 may be retained by the second filter FP2, and thus may not be removed from the primary refined material PM1.

The second filter FP2 used in the second refining process 5300 may be filled with the second filler AD12. In an embodiment, the second filter FP2 may be further filled with the first filler AD11 in the course of the second refining process 5300. For example, in the second mixture OM2, the impurities IP may remain adsorbed onto the first filler AD11. Accordingly, when the second mixture OM2 is put into the second filter FP2, the second filter FP2 may be filled with the first filler AD11, in addition to the second filler AD12.

The second filler AD12 may correspond to the filler AD described above with reference to any of FIGS. 6A to 6C. The contents of the second filler AD12 may be the same as the contents of the filler AD according to one of FIGS. 6A to 6C. For example, the second filler AD12 may include at least one of diatomaceous earth, white clay, and red clay. For example, the second filler AD12 may include diatomaceous earth, but embodiments are not limited thereto.

The second filler AD12 in the second filter FP2 may be the same as or different from the first filler AD11. For example, the first filler AD11 and the second filler AD12 may each include diatomaceous earth. FIG. 7C shows that the first filler AD11 and the second filler AD12 are the same material. However, embodiments are not limited thereto, and the first filler AD11 and the second filler AD12 may include different materials.

Whether the impurities IP and/or the host material HM remain in the secondary refined material PM2 that passes through the second filter FP2 in the course of the second refining process S300 may be determined, for example, by visual means or by an analytical process. For example, the secondary refined material PM2 that passes through the second filter FP2 may be examined by using the naked eye and/or by thin layer chromatography (TLC). Accordingly, when no impurities IP and/or host material HM remain in the secondary refined material PM2, the secondary refined material PM2 may be dried to obtain a high purity doping material DM. The doping material DM obtained after the second refining process S300 may have a purity equal to or greater than about 99.5%. Accordingly, the doping material DM refined through the method for refining an organic material according to an embodiment may be recycled.

In embodiments, when it is determined that the secondary refined material PM2 obtained through the second refining process S300 contains the impurities IP, a third refining process may be further performed after the second refining process S300. For example, the obtained secondary refined material PM2 may contain some impurities IP such as ash. Accordingly, a third refining process may be performed to remove ash from the secondary refined material PM2.

In an embodiment, in the third refining process, the secondary refined material PM2 containing the impurities IP and doping material DM may be mixed with a third organic solvent. At least a portion of the secondary refined material PM2 containing the impurities IP and doping material DM may be dissolved by the third organic solvent. For example, the impurities IP such as ash included in the secondary refined material PM2 may be dissolved in the third organic solvent.

In an embodiment, the third organic solvent may include ethyl acetate. When the secondary refined material PM2 containing the impurities IP and doping material DM is mixed with ethyl acetate, the impurities IP may be dissolved by the third organic solvent, while the doping material DM may not be dissolved in the ethyl acetate. Therefore, after mixing the secondary refined material PM2 containing the impurities IP and doping material DM with the third organic solvent, the mixture (hereinafter referred to as a third mixture) may be filtered through a filter to remove residual impurities IP from the secondary refined material PM2.

The filter used in the third refining process may be referred to as a third filter. The third filter may be different from the first filter FP1 and second filter FP2 as described above. The third filter may not include the first filler AD11 or the second filler AD12 as described above. For example, the third filter may be a filter of the related art that filters materials that are not dissolved by the third organic solvent, from among the materials included in the third mixture.

In the third refining process, when the third mixture is filtered through the third filter, the impurities IP dissolved by the third organic solvent may pass through the third filter, and the doping material DM may remain inside the third filter. The doping material DM remaining in the third filter may be recycled after drying.

In an embodiment, the method for refining an organic material may further include the third refining process and thus remove the impurities IP remaining in the secondary refined material PM2, thereby obtaining a high purity doping material DM.

In embodiments, when it is determined that the secondary refined material PM2 obtained through the second refining process S300 contains the host material HM, a fourth refining process may be further performed after the second refining process S300. For example, the obtained secondary refined material PM2 may contain a portion of the host material HM. Accordingly, a fourth refining process may be performed to remove the host material HM from the secondary refined material PM2.

The fourth refining process may be performed in a substantially same manner as the first refining process S200 as described above. In the fourth refining process, the description of the first filter FP1 and the first organic solvent, as described with reference to FIG. 7A, may be applied to the fourth refining process in a substantially similar manner.

In an embodiment, in the fourth refining process, the host material HM may be separated from the secondary refined material PM2 by using the first filter FP1. In the fourth refining process, the host material HM remaining in the secondary refined material PM2 may be removed by using the first filter FP1.

For example, in the fourth refining process, the secondary refined material PM2 containing the host material HM may be mixed with the first organic solvent as described above. As described above, the first organic solvent may have a pH in a range of about 3.0 to about 4.0. In the fourth refining process, the secondary refined material PM2 and the first organic solvent may be mixed at a volume ratio of about 1:5 (secondary refined material:first organic solvent), but embodiments are not limited thereto. The residual host material HM included in the secondary refined material PM2 may be dissolved by the first organic solvent.

In the fourth refining process, when the mixture of the secondary refined material PM2 and the first organic solvent is filtered through the first filter FP1, the secondary refined material PM2 excluding the host material HM, such as the doping material DM, may be filtered by the first filler AD11 in the first filter FP1. The host material HM may pass through the first filter FP1. In the fourth refining process, whether the host material HM remains in the secondary refined material PM2 may be determined by using thin layer chromatography (TLC), as described in the first refining process S200.

In the fourth refining process, the secondary refined material PM2 that is not dissolved by the first organic solvent may be adsorbed onto the first filler AD11. For example, the doping material DM that is not dissolved by the first organic solvent and adsorbed onto the first filler AD11 may remain inside the first filter FP1. Accordingly, an additional process may be performed to separate the doping material DM adsorbed onto the first filler AD11.

In an embodiment, the doping material DM adsorbed onto the first filler AD11 may be placed in a reactor and a solvent such as acetonitrile may be added to separate the doping material DM from the first filler AD11. For example, when the doping material DM adsorbed onto the first filler AD11 is mixed with acetonitrile, the doping material DM may be dissolved in acetonitrile. Accordingly, when the mixture of the first filler AD11, onto which the doping material DM is adsorbed, and acetonitrile is filtered through a filter of the related art, the first filler AD11 may be filtered, and the doping material DM separated from the first filler AD11 may be obtained. Thereafter, the doping material DM may be dried to obtain a high purity refined material.

In an embodiment, the fourth refining process may be performed between the second refining process 5300 and the third refining process, but embodiments are not limited thereto. For example, the fourth refining process may be performed after sequentially performing the first refining process, the second refining process, and the third refining process.

Referring again to FIGS. 3 and 4, the emission layer EML may be provided on the hole transport region HTR. The emission layer EML may have a thickness in a range of about 100 Å to about 1,000 Å. For example, the emission layer EML may have a thickness in a range of about 100 Å to about 300 Å. The emission layer EML may have a structure consisting of a layer consisting of a single material, a structure consisting of a layer including different materials, or a structure including multiple layers including different materials.

In the light emitting element ED, the emission layer EML may include an anthracene derivative, a pyrene derivative, a fluoranthene derivative, a chrysene derivative, a dihydrobenzanthracene derivative, or a triphenylene derivative. for example, the emission layer EML may include an anthracene derivative or a pyrene derivative. In the light emitting element ED according to an embodiment, the emission layer EML may include a host and a dopant.

In the light emitting element ED according to embodiments as shown in FIGS. 3 and 4, an electron transport region ETR may be provided on the emission layer EML. The electron transport region ETR may include at least one of a hole blocking layer (not shown), an electron transport layer ETL, and an electron injection layer EIL, but embodiments are not limited thereto.

The electron transport region ETR may have a structure consisting of a layer consisting of a single material, a structure consisting of a layer including different materials, or a structure including multiple layers including different materials.

For example, the electron transport region ETR may have a single-layered structure of an electron injection layer EIL or an electron transport layer ETL, or may have a single-layered structure formed of an electron injection material and an electron transport material. The electron transport region ETR may have a single-layered structure formed of different materials. In embodiments, the electron transport region ETR may have an electron transport layer ETL/electron injection layer EIL structure, or a hole blocking layer (not shown)/electron transport layer ETL/electron injection layer EIL structure, in which the layers of each structure may be stacked in its respective stated order from the emission layer EML, but embodiments are not limited thereto. The electron transport region ETR may have a thickness, for example, in a range of about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

In embodiments, the electron transport region ETR may include: metal halides such as LiF, NaCl, CsF, RbCl, RbI, Cul, and KI; lanthanide metals such as Yb, or a co-deposited material of a metal halide and a lanthanide metal. For example, the electron transport region ETR may include KJ:Yb, RbJ:Yb, LiF:Yb, or the like as a co-deposited material. The electron transport region ETR may include a metal oxide such as Li₂O and BaO, or 8-hydroxyl-lithium quinolate (Liq), or the like, but embodiments are not limited thereto. In another embodiment, the electron transport region ETR may include a mixture of an electron transport material and an insulating organometallic salt. The organometallic salt may be a material having an energy band gap equal to or greater than about 4 eV. For example, the organometallic salt may include a metal acetate, a metal benzoate, a metal acetoacetate, a metal acetylacetonate, or a metal stearate.

The electron transport region ETR may further include, for example, at least one of 2,9-dimethyl-4,7-diphenyl-1,10-phenanthroline (BCP), diphenyl(4-(triphenylsilyl)phenyl)phosphine oxide (TSPO1), 2-phenyl-4,6-bis(3-(triphenylsilyl)phenyl)-1,3,5-triazine, and 4,7-diphenyl-1,10-phenanthroline (Bphen), in addition to the materials described above, but embodiments are not limited thereto.

The electron transport region ETR may include the compounds of the electron transport region ETR as described above in at least one of an electron injection layer EIL, an electron transport layer ETL, and a hole blocking layer (not shown).

When the electron transport region ETR includes an electron transport layer ETL, the electron transport layer ETL may have a thickness in a range of about 100 Å to about 1,000 Å. For example, the electron transport layer ETL may have a thickness in a range of about 150 Å to about 500 Å. When the thickness of the electron transport layer ETL satisfies any of the above-described ranges, satisfactory electron transport properties may be obtained without a substantial increase in driving voltage. When the electron transport region ETR includes an electron injection layer EIL, the electron injection layer EIL may have a thickness in a range of about 1 Å to about 100 Å. For example, the electron injection layer EIL may have a thickness in a range of about 3 Å to about 90 Å. When the thickness of the electron injection layer EIL satisfies any of the above-described ranges, satisfactory electron injection properties may be obtained without a substantial increase in driving voltage.

The second electrode EL2 may be provided on the electron transport region ETR. The second electrode EL2 may be a common electrode. The second electrode EL2 may be a cathode or an anode but embodiments are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may include a transparent metal oxide, for example, indium tin oxide (ITO), indium zinc oxide (IZO), zinc oxide (ZnO), indium tin zinc oxide (ITZO), or the like.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgYb). In an embodiment, the second electrode EL2 may have a multilayered structure that includes a reflective film or a transflective film formed of the above-described materials, and a transparent conductive film formed of indium tin oxide (ITO), indium zinc oxide (IZO), zinc oxide (ZnO), indium tin zinc oxide (ITZO), or the like. For example, the second electrode EL2 may include the above-described metal materials, a combination of two or more of the above-described metal materials, or oxides of the above-described metal materials.

Although not shown in the drawings, in an embodiment, the second electrode EL2 may be electrically connected to an auxiliary electrode. When the second electrode EL2 is electrically connected to an auxiliary electrode, resistance of the second electrode EL2 may decrease.

In an embodiment, the light emitting element ED may further include a capping layer disposed on the second electrode EL2. The capping layer may have a multilayered structure or a single-layered structure.

In an embodiment, the capping layer may include an organic layer or an inorganic layer. For example, when the capping layer includes an inorganic material, the inorganic material may include an alkali metal compound such as LiF, an alkaline earth metal compound such as MgF₂, SiON, SiNₓ, or SiO_{y}.

For example, when the capping layer includes an organic material, the organic material may include α-NPD, NPB, TPD, m-MTDATA, Alq₃ CuPc, N4,N4,N4′,N4′-tetra(biphenyl-4-yl) biphenyl-4,4′-diamine (TPD15), 4,4′,4″-tris(carbazol-9-yl)triphenylamine (TCTA), etc., or may include an epoxy resin, or an acrylate such as methacrylate. However, embodiments are not limited thereto, and the capping layer may include at least one of Compounds P1 to P5:

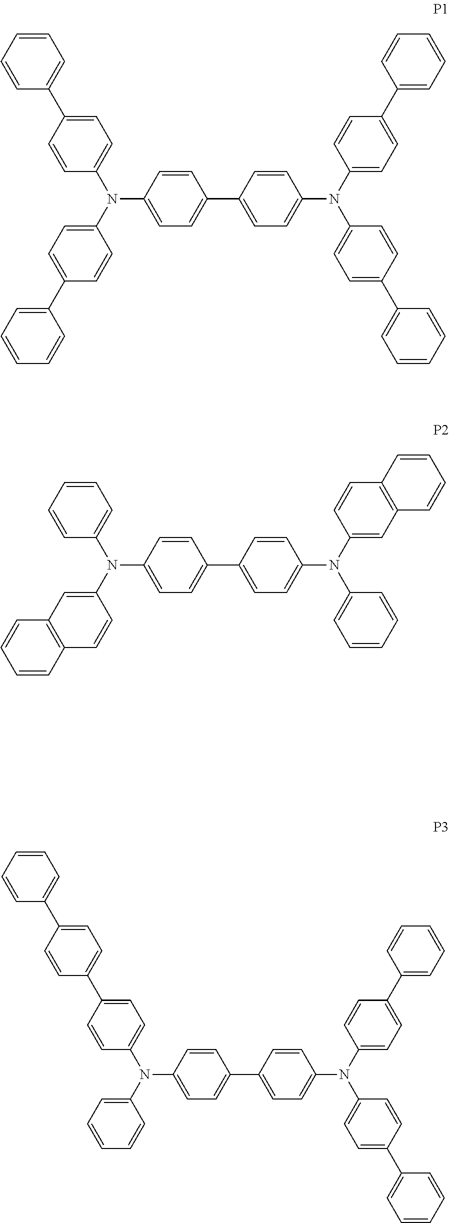 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

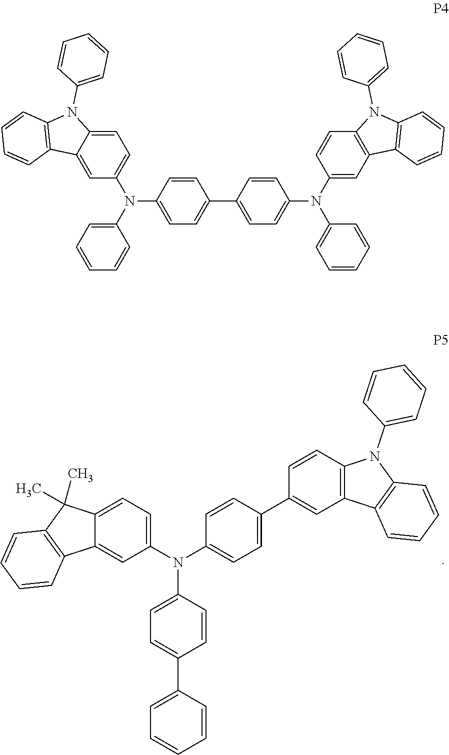 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

The capping layer may have a refractive index equal to or greater than about 1.6. For example, the capping layer may have a refractive index equal to or greater than about 1.6 in a wavelength range of about 550 nm to about 660 nm.

Using the method for refining an organic material according to an embodiment may allow a doping material to be efficiently and quickly refined to high purity from an organic material recovered from deposition equipment, a crucible, or the like, after a light emitting element deposition process. For example, a p-dopant used in the deposition of a hole injection layer included in a light emitting element may be refined to high purity. The doping material refined through the method for refining an organic material according to an embodiment may have a purity equal to or greater than about 99.5%, and the doping material may thus be recycled, thereby providing substantial cost savings.

In embodiments, an electronic device may include a display device including a multiple light-emitting elements, and a control part that controls the display device. At least one of the light-emitting elements may include an organic material that is separated and refined through the method for refining an organic material according to embodiments. For example, at least one of the light-emitting elements may include the organic material that is separated and refined through the method for refining an organic material according to an embodiment in a hole transport region. The electronic device according to embodiments may be a device that is activated according to an electrical signal. The electronic device may include one or mor display devices according to embodiments. Examples of an electronic device may include small, medium-sized, and large electronic devices such as a television set, a monitor, a billboard, a personal computer, a laptop computer, a personal digital terminal, a display device for a vehicle, a game console, a portable electronic device, and a camera.

Hereinafter, a method for refining an organic material according to an embodiment will be described with reference to the Examples and the Comparative Examples. The Examples described below are only provided to assist in understanding the disclosure, and the scope thereof is not limited thereto.

### EXAMPLES

### 1. Refining of Contaminated Organic Materials

### 1) Recovery of Contaminated Organic Materials

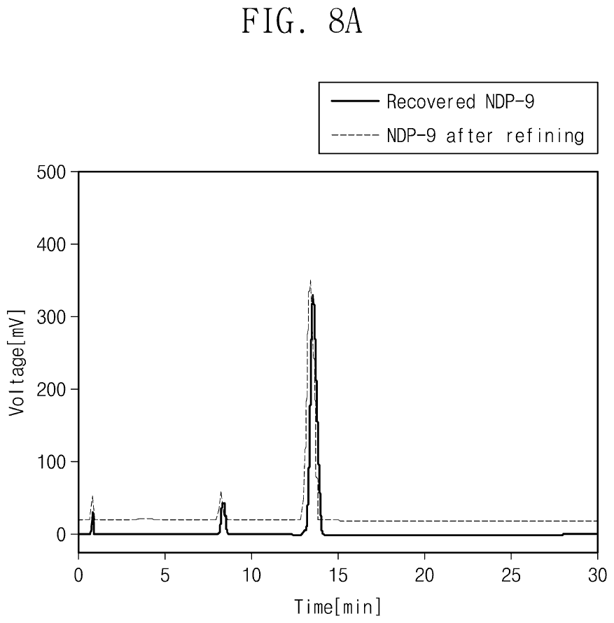

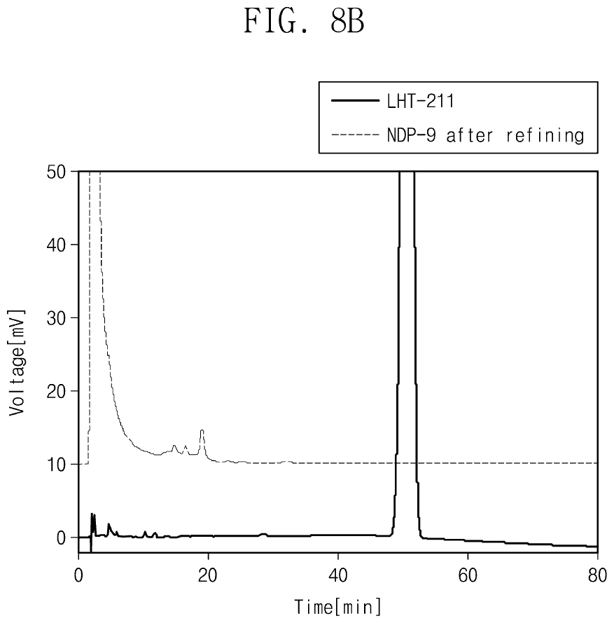

After a light emitting element deposition process, 4-[[2,3-bis[cyano-(4-cyano-2,3,5,6-tetrafluorophenyl)methylidene]cyclopropylidene]-cyanomethyl]-2,3,5,6-tetrafluorobenzonitrile (hereinafter referred to as NDP-9) as a doping material was recovered and collected from a crucible, a deposition plate, or the like. The recovered NDP-9 was analyzed through liquid chromatography in analysis conditions shown in Tables 1 and 2. The results are shown in Table 3, Table 4, and FIG. 8A. Table 1 shows the analysis conditions for the recovered NDP-9, and Table 2 shows the conditions for determining whether a host material of LHT-211 is mixed in the recovered NDP-9.

TABLE 1

| | |
|---|---|
| Column | YMC-Triart C18, 3.0 × 100 mm, 5 μm |

| | | |
|---|---|---|
| Flow rate | 0.56 | mL/min |
| Detection wavelength | 490 | nm |

| | |
|---|---|
| Mobile phase | Acetonitrile |

| | | |
|---|---|---|
| Temperature | 35° | C. |

TABLE 2

| | |
|---|---|
| Column | Agilent ZORBAX Eclipse XDB-C18, |
| | 4.6 × 250 mm, 5 μm |

| | | |
|---|---|---|
| Flow rate | 1 | mL/min |
| Detection wavelength | 254 | nm |

| | |
|---|---|
| Mobile phase | Tetrahydrofuran and Distilled water |

| | | |
|---|---|---|
| Temperature | 35° | C. |

In Tables 3 and 4, ‘before refining’ is the result of analyzing the recovered NDP-9 through liquid chromatography without refining. Referring to the results of ‘before refining’ in Tables 3 and 4, peaks 1 to 7 indicate that unrefined NDP-9 contained various impurities from thermal decomposition and other materials contaminated during the work process. In Tables 3 and 4, peak 8 indicates that the unrefined NDP-9 had a purity of 99.127%, and LHT-211 was mixed in the unrefined NDP-9.

### 2) Example 1

The recovered NDP-9 (hereinafter referred to as a contaminated organic material), which was found to be mixed with impurities and the host material of LHT-211, was refined using the method for refining an organic material according to an embodiment. 10 g of contaminated organic material was added to 1 L of methylene chloride buffered at a pH of 3.75, using formic acid and ammonia, and the mixture was stirred for 3 hours. The first mixture of partially dissolved contaminated organic material and organic solvent (formic acid, ammonia, methylene chloride) was filtered through a first filter filled with diatomaceous earth.

For the solution filtered through the first filter in the first mixture, the presence or absence of LHT-211 was determined using thin layer chromatography (TLC) from the moment the solution passed through the first filter, and filtration was performed until it was determined that LHT-211 was no longer present. A primary refined material of diatomaceous earth and the material adsorbed onto the diatomaceous earth in the first filter was placed in a reactor, and 50 times the amount of acetonitrile was added and the mixture was stirred for 2 hours to obtain a second mixture.

The second mixture was placed in a second filter filled with diatomaceous earth and filtered. When filtering the second mixture, for the solution filtered through the second filter, the presence or absence of impurities was determined using thin layer chromatography (TLC) from the moment the solution passed through the second filter, and a secondary refined material was obtained from the second mixture with impurities removed.

In order to remove ash that remained in the secondary refined material, the secondary refined material is partially dissolved in 3 times the volume of ethyl acetate, and filtered through a third filter capable of filtering out undissolved materials. The material filtered through the third filter was dried to obtain 7.1 g of refined doping material NDP-9. The refined NDP-9 was analyzed through liquid chromatography in analysis conditions shown in Tables 1 and 2, and the results are shown in Table 3 and FIG. 8A. In Table 3, ‘after refining’ is the result of analyzing the refined NDP-9, using liquid chromatography.

TABLE 3

| | |
|---|---|
| Before refining | After refining |

| | Retention | | Retention | |
|---|---|---|---|---|
| Peak | Time (min) | % Area | Time (min) | % Area |
| 1 | 4.0 | 0.051 | 4.0 | 0.014 |
| 2 | 10.0 | 0.011 | 10.0 | 0.005 |
| 3 | 11.0 | 0.009 | 11.0 | X |
| 4 | 11.6 | 0.009 | 11.6 | X |
| 5 | 12.0 | 0.205 | 12.0 | 0.069 |
| 6 | 13.0 | 0.574 | 13.0 | X |
| 7 | 14.4 | 0.014 | 14.4 | 0.032 |
| 8 | 14.8 | 99.127 | 14.8 | 99.879 |

| | | |
|---|---|---|
| LHT-211 | 26.4% | X |

Referring to Table 3 and FIG. 8A, it is determined that NDP-9 refined through the method for refining an organic material according to an embodiment showed a high purity equal to or greater than 99.5%. It is determined that NDP-9 recovered through the method for refining an organic material according to an embodiment showed a high purity of 99.879% as the impurities observed at peak 3, peak 4, and peak 6 were removed, as compared to before refining.

The results of liquid chromatography analysis of LHT-211 and NDP-9 refined through the method of Example 1 are shown in FIG. 8B. Referring to Table 3 and FIG. 8B, it is determined that NDP-9 refined through the method of Example 1 contained no host material of LHT-211, as compared to before refining. As determined in FIG. 8B, the refined NDP-9 had no peak observed at the same position as LHT-211, indicating that the refined NDP-9 contained no host material.

### 3) Comparative Example 1 and Comparative Example 2

The contaminated organic material was refined through the method of Comparative Examples 1 and 2. Comparative Example 1 is a method of refining NDP-9 by mixing the contaminated organic material with methylene chloride and merely filtering the mixture through a filter filled with a silica gel filler, and Comparative Example 2 is a method of refining NDP-9 by mixing the contaminated organic material with methylene chloride and merely filtering the mixture through a filter filled with an alumina filler.

The NDP-9 refined by using the method of Comparative Examples 1 and 2 was each analyzed by liquid chromatography in analysis conditions shown in Tables 1 and 2 above, and the results are shown in Table 4.

TABLE 4

| | Before refining | | Comparative Example 1 | | Comparative Example 2 | |
|---|---|---|---|---|---|---|
| | Retention | | Retention | | Retention | |
| Peak | Time (min) | % Area | Time (min) | % Area | Time (min) | % Area |
| 1 | 4.0 | 0.051 | 4.0 | X | 4.0 | 0.003 |
| 2 | 10.0 | 0.011 | 10.0 | 0.005 | 10.0 | 0.010 |
| 3 | 11.0 | 0.009 | 11.0 | 0.007 | 11.0 | 0.008 |
| 4 | 11.6 | 0.009 | 11.6 | 0.008 | 11.6 | 0.009 |
| 5 | 12.0 | 0.205 | 12.0 | 0.196 | 12.0 | 0.198 |
| 6 | 13.0 | 0.574 | 13.0 | 0.561 | 13.0 | 0.541 |
| 7 | 14.4 | 0.014 | 14.4 | 0.057 | 14.4 | 0.061 |
| 8 | 14.8 | 99.127 | 14.8 | 99.166 | 14.8 | 99.169 |

| | | | |
|---|---|---|---|
| LHT-211 | 26.4% | 21.8% | 25.3% |

Referring to Table 4, it is determined that most of the impurities identified at peaks 1 to 7 were not removed from NDP-9 refined using the refining methods of Comparative Examples 1 and 2. Accordingly, it is determined that the refined NDP-9 of Comparative Examples 1 and 2 showed a purity of less than 99.5%, which is not suitable for a recycling process. It is determined that the host material of LHT-211 was not completely removed and remained in NDP-9 that was refined according to the refining methods of Comparative Examples 1 and 2.

Using a method for refining an organic material according to an embodiment may allow a doping material to be efficiently and quickly refined to high purity from an organic material recovered from deposition equipment, a crucible, or the like, after a light emitting element deposition process. Accordingly, expensive doping materials may be recycled.

The method for refining an organic material according to an embodiment uses a filter that is filled with a particular filler when separating a doping material from a recovered organic material and thus may provide substantial cost savings in manufacturing a display device containing a doping material.

The method for refining an organic material according to an embodiment may be used even when a target material for refining is a material having low solubility in a solvent. The material having low solubility in a solvent may have a solubility of about 3 to about 5 times greater when the method for refining an organic material according to an embodiment is applied. For example, the material may have a solubility of about 5 times or greater when using the method for refining an organic material according to an embodiment.

Embodiments have been disclosed herein, and although terms are employed, they are used and are to be interpreted in a generic and descriptive sense only and not for the purposes of limitation. In some instances, as would be apparent to one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A method for refining an organic material, the method comprising:
a recovery process of recovering a contaminated organic material from a light emitting element deposition process;
a first refining process comprising:
mixing the contaminated organic material, which includes impurities, material A, and material B, with a first organic solvent having a pH in a range of about 3.0 to about 4.0; and
filtering the mixture through a first filter to separate a primary refined material, which includes the impurities and material B, from the contaminated organic material; and
a second refining process comprising:
mixing the primary refined material with a second organic solvent that is different from the first organic solvent; and
filtering the mixture through a second filter to separate a secondary refined material, which includes material B, from the primary refined material, wherein
the first filter is filled with a first filler,
the second filter is filled with a second filler, and
the first filler and the second filler each independently include at least one of diatomaceous earth, white clay, and red clay.

2. The method of claim 1, wherein the first organic solvent comprises methylene chloride and a buffer solution.

3. The method of claim 2, wherein the buffer solution comprises formic acid and ammonia.

4. The method of claim 1, wherein the second organic solvent comprises acetonitrile.

5. The method of claim 1, wherein in the first refining process,
material A is dissolved by the first organic solvent and passes through the first filter, and
the primary refined material is adsorbed onto the first filler.

6. The method of claim 5, wherein in the second refining process,
the primary refined material, adsorbed onto the first filler, is mixed with the second organic solvent,
material B in the primary refined material is dissolved by the second organic solvent,
the dissolved material B passes through the second filter and is separated with the secondary refined material, and
the impurities in the primary refined material are adsorbed onto the second filler.

7. The method of claim 1, further comprising:
a third refining process comprising:
mixing the secondary refined material with a third organic solvent to dissolve residual impurities; and
filtering the mixture to remove the residual impurities from the secondary refined material, wherein
the residual impurities are the impurities that remain in the secondary refined material after the second refining process.

8. The method of claim 7, wherein the third organic solvent comprises ethyl acetate.

9. The method of claim 1, further comprising:
a fourth refining process comprising:
mixing the secondary refined material with the first organic solvent; and
refiltering the mixture through the first filter to remove residual material A from
the secondary refined material, wherein
the residual material A is the material A that remains in the secondary refined material after the second refining process.

10. The method of claim 9, wherein in the fourth refining process,
the secondary refined material is adsorbed onto the first filler, and
the residual material A is dissolved in the first organic solvent and passes through the first filter.

11. The method of claim 1, wherein the first filler and the second filler each include diatomaceous earth.

12. The method of claim 1, wherein
the first filler and the second filler each include diatomaceous earth, white clay, and red clay, and
in each of the first filler and the second filler, an amount of the diatomaceous earth is greater than a total amount of the white clay and the red clay.

13. The method of claim 1, wherein material B is a p-dopant material.

14. The method of claim 13, wherein the p-dopant material comprises a cyano group-containing compound.

15. The method of claim 14, wherein the cyano group-containing compound comprises a compound represented by Formula 1:
[Formula 1] 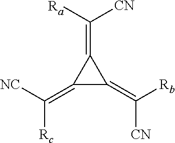
wherein in Formula 1,
Rₐ, R_{b}, and R_{c} are each independently a cyano group, a halogen atom, a substituted or unsubstituted alkyl group having 1 to 20 carbon atoms, a substituted or unsubstituted hydrocarbon ring having 3 to 60 ring-forming carbon atoms, or a substituted or unsubstituted heterocycle having 2 to 60 ring-forming carbon atoms, and
at least one of Rₐ, R_{b}, and R_{c} is each independently: a cyano group; —F; —Cl; —Br; —I; an alkyl group having 1 to 20 carbon atoms substituted with a cyano group, —F, —Cl, —Br, —I, or a combination thereof; a hydrocarbon ring having 3 to 60 ring-forming carbon atoms substituted with a cyano group, —F, —Cl, —Br, —I, or a combination thereof; or a heterocycle having 2 to 60 ring-forming carbon atoms substituted with a cyano group, —F, —Cl, —Br, —I, or a combination thereof.

16. The method of claim 15, wherein the cyano group-containing compound comprises a compound represented by Formula A: 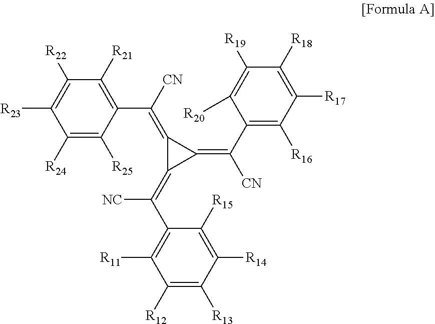
wherein in Formula A,
at least one of R₁₁ to R₂₅ is each independently a fluorine atom, and
the remainder of Rn to R₂₅ are each independently a hydrogen atom, a tritium atom, a fluorine atom, a cyano group, a substituted or unsubstituted alkyl group having 1 to 30 carbon atoms, or a substituted or unsubstituted alkenyl group having 2 to 30 carbon atoms.

17. The method of claim 1, wherein the first organic solvent has a pH in a range of about 3.7 to about 3.8.

18. The method of claim 1, wherein the material B obtained after the second refining process has a purity equal to or greater than about 99.5%.

19. A method for refining an organic material, the method comprising:
recovering a contaminated organic material including impurities, material A, and material B from a light emitting element deposition process;
separating and refining a primary refined material that includes impurities and material B from the contaminated organic material, by using a first organic solvent having a pH in a range of about 3.0 to about 4.0; and
separating and refining a secondary refined material that includes material B from the primary refined material, by using a second organic solvent that is different from the first organic solvent.

20. An electronic device comprising:
a display device, the display device comprising:
a circuit layer disposed on a base layer;
a pixel defining film disposed on the circuit layer and having a plurality of pixel openings defined therein; and
a plurality of light emitting elements disposed on the circuit layer, wherein
at least one of the light emitting elements comprises an organic material that is separated and refined through a method for refining an organic material, the method comprising:
a first refining process comprising:
mixing a contaminated organic material, which includes impurities, material A, and material B, with a first organic solvent having a pH in a range of about 3.0 to about 4.0; and
filtering the mixture through a first filter to separate a primary refined material, which includes the impurities and material B, from the contaminated organic material; and
a second refining process comprising:
mixing the primary refined material with a second organic solvent that is different from the first organic solvent; and
filtering the mixture through a second filter to separate a secondary refined material, which includes material B, from the primary refined material, wherein
the first filter is filled with a first filler,
the second filter is filled with a second filler, and
the first filler and the second filler each independently include at least one of diatomaceous earth, white clay, and red clay.
